(19) United States
(12) Patent Application Publication
(10) Pub. No.: US 2021/0100837 A1
(43) Pub. Date: Apr. 8, 2021

(54) TREATMENT OF FIBROSIS WITH GENETICALLY-ENGINEERED MACROPHAGES

(71) Applicant: The University of Chicago, Chicago, IL (US)

(72) Inventors: Xuewen GOU, Chicago, IL (US); Yingming ZHAO, Chicago, IL (US); Jianfeng DU, Chicago, IL (US); Xiaoyang WU, Chicago, IL (US); Jiping YUE, Chicago, IL (US); Lev BECKER, Chicago, IL (US)

(21) Appl. No.: 15/733,201

(22) PCT Filed: Dec. 14, 2018
(86) PCT No.: PCT/US18/65773

§ 371 Date: Jun. 9, 2020

Related U.S. Application Data
(60) Provisional application No. 62/598,894, filed on Dec. 14, 2017.

(51) Int. Cl. A61K 35/15; A61P 19/04; C07K 14/705; C12N 9/64

(52) U.S. Cl. CPC A61K 35/15; A61P 19/04; A61K 9/0019; C12N 9/6491; C12Y 304/24007; C07K 14/7055

(57) ABSTRACT

Provided herein are macrophages engineered for treating fibrosis and ameliorating the effects of fibrotic lesions in various organs and tissues. Certain embodiments are directed to genetically-engineered macrophages capable of treating fibrosis or reducing fibrotic lesions. In certain aspects macrophages can be genetically-engineered to (1) target extracelluar matrix (ECM) or components thereof, (2) enhance degradation of ECM, or (3) target ECM and enhance degradation of ECM. Further provided is a cellular therapy product comprising a genetically-engineered macrophage comprising at least one of a recombinant targeting protein and a recombinant catalytic enzyme. Further provided is a method of treating an individual for fibrosis comprising administering the cellular therapy product.

### CROSS-REFERENCE TO RELATED APPLICATIONS

This application claims the benefit of priority of U.S. Provisional Patent Application No. 62/598,894 filed Dec. 14, 2017, which is hereby incorporated by reference in its entirety.

### STATEMENT REGARDING FEDERALLY SPONSORED RESEARCH

This invention was made with government support under grant numbers R01 OD023700 and R01 DK102960 each awarded by the National Institutes of Health. The government has certain rights in the invention.

### BACKGROUND OF THE INVENTION

### Field of the Invention

This disclosure relates generally to the fields of molecular biology and medicine; in particular to genetically-engineered macrophages and their use in the treatment of fibrosis.

Description of Related Art

Fibrosis is the common scarring reaction associated with chronic injury that results from prolonged parenchymal cell injury and/or inflammation that may be induced by a wide variety of agents, e.g., drugs, toxins, radiation, any process disturbing tissue or cellular homeostasis, toxic injury, altered blood flow, infections (viral, bacterial, spirochetal, and parasitic), storage disorders, and disorders resulting in the accumulation of toxic metabolites. Fibrosis is most common in the liver, heart, lung, peritoneum, and kidney.

For instance, hepatic fibrosis (liver fibrosis) results from an altered wound healing response that is characterized by increased production of matrix proteins and decreased matrix remodeling. Normal structural elements of tissues are replaced with excessive amounts of non-functional scar tissue. Hepatic fibrosis is a common pathological consequence of chronic liver diseases. In a number of patients, fibrosis ultimately leads to cirrhosis, a condition defined by an abnormal liver architecture, with fibrotic septa surrounding regenerating nodules and altered vacularization. Due to decreased functional parenchymal reserve and altered hepatic blood flow, cirrhosis is associated with the life-threatening complications of liver failure including hepatic encephalopathy, coagulation disorders and bacterial infections, and complications of portal hypertension such as ascites, variceal rupture and hepatorenal syndrome. In addition, the cirrhotic liver is a precancerous state, and thus requires the systematic screening for hepatocellular carcinoma. Several clinical reports have documented that regression of liver fibrosis occurs in a substantial proportion of patients, provided that the factor responsible for liver insult is eradicated or controlled. Consistent with this observation, studies in rodents have also documented regression of fibrosis or early stage cirrhosis within weeks following eradication of the toxic insult. The potential for reversibility of fibrosis declines at advanced stages. It is imperative to treat fibrosis in the early stages of reversible liver scarring so that irreversible cirrhosis can be prevented.

There remains a need for additional and/or improved therapies to reverse fibrosis in individuals suffering from fibrotic conditions.

### SUMMARY OF THE INVENTION

Embodiments described herein provide macrophages engineered for treating fibrosis and ameliorating the effects of fibrotic lesions in various organs and tissues. Certain embodiments are directed to genetically-engineered macrophages capable of treating fibrosis or reducing fibrotic lesions. In certain aspects macrophages can be genetically-engineered to (1) target extracelluar matrix (ECM) or components thereof, (2) enhance degradation of ECM, or (3) target ECM and enhance degradation of ECM. Macrophages can be engineered to target ECM by expressing one or more cell surface receptors (e.g., a collagen receptor) that bind one or more component of the ECM (e.g., collagen). In addition, macrophages can be engineered for enhance degradation of ECM by expression of a protease or other enzyme that cleaves or degrades one or more ECM component (e.g., matrix metalloprotease, MMP).

In a first aspect, a genetically-engineered macrophage can include or express a recombinant targeting protein and/or a recombinant catalytic enzyme. A recombinant targeting protein can include a protein that binds an ECM component, e.g., collagen. In one embodiment, the recombinant targeting protein is a collagen receptor or a subunit thereof. Collagen receptors are membrane proteins that bind the extracellular matrix protein collagen. In one embodiment, the collagen receptor or a subunit thereof comprises one or more of an integrin, a discoidin domain receptor, a mannose family receptor, and/or an immunoglobulin-like receptor. The integrin can be a α1β1, α2β1, α10β1, and/or α11β1 integrin. In one aspect, the discoidin domain receptor (DDR) can be DDR1 and/or DDR2. In one aspect, the mannose family receptor can be M-phospholipase A2 receptor and/or Endo180. In one aspect, the immunoglobulin-like receptor can be glycoprotein VI.

In one aspect, the recombinant targeting protein is Integrin Alpha 1 (ITGA-1) (SEQ ID NO: 1 or SEQ ID NO: 3, mouse (e.g., GenBank accession number NP_001028400.2) and human (e.g., GenBank accession number NP_852478.1, respectively). In certain aspects a macrophage can express a nucleic acid that is or is at least 70, 75, 80, 85, 90, 91, 92, 93, 94, 95, 96, 97, 98, 99, or 100% identical to SEQ ID NO:1 or SEQ ID NO:3 (or any range derivable therein), or a segment thereof. In other aspects a macrophage can express a polypeptide that is or is at least 80, 85, 90, 91, 92, 93, 94, 95, 96, 97, 98, 99, or 100% identical to SEQ ID NO:2 or SEQ ID NO:4 (or any range derivable therein), or a functional variant or segment thereof.

SEQ ID NO:1 provides the full length coding sequence of mouse ITGA-1 that encodes the amino acid sequence of SEQ ID NO:2. SEQ ID NO:2 is a 1179 amino acid protein having a signal peptide from amino acid 1 to 28 (mature protein comprising amino acids 29 to 1179 of SEQ ID NO:2) and a transmembrane region from approximately amino acid 1142 to 1164 of SEQ ID NO:2. In certain aspects a segment of SEQ ID NO:2 can be expressed by an engineered macrophage, the segment comprising, comprising at least, or comprising at most 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179 contiguous amino acids of SEQ ID NO:2 (or any range therein) starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679 of SEQ ID NO2 (or any range of positions therein), and ending at amino acid 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, or 1179 of SEQ ID NO:2 (or any range of positions therein).

SEQ ID NO:3 provides the full length coding sequence of human ITGA-1 that encodes the amino acid sequence of SEQ ID NO:4. SEQ ID NO:4 is a 1179 amino acid protein having a signal peptide from amino acid 1 to 28 (mature protein comprising amino acids 29 to 1179 of SEQ ID NO:4) and a transmembrane region from approximately amino acid 1142 to 1164 of SEQ ID NO:4. In certain aspects a segment of SEQ ID NO:4 can be expressed by an engineered macrophage, the segment comprising, comprising at least, or comprising at most 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179 amino acids of SEQ ID NO:4 (or any range derivable therein) starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679 of SEQ ID NO:4 (or any range of positions therein), and ending at amino acid 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, or 1179 of SEQ ID NO:4 (or any range of positions therein).

In one embodiment, the recombinant catalytic enzyme is a protease. In certain aspects the protease is a matrix metalloproteinase (MMP). In one embodiment, the matrix metalloproteinase is MMP1 (e.g., NP_002412), MMP1a (e.g., NP_114395.1), MMP2 (e.g., NP_001121363), MMP3 (e.g., NP_002413), MMPI (e.g., NP_002414), MMP8 (e.g., NP_001291370), MMP9 (e.g., NP_004985), MMP10 (e.g., NP_002416), MMP12 (e.g., NP_002417), MMP13 (e.g., NP_002418), MMP14 (e.g., NP_004986), MMP17 (e.g., NP_057239), MMP19 (e.g., NP_001259030), MMP20 (e.g., NP_004762), MMP21 (e.g., NP_671724), MMP22 (NP_008914.1), MMP24 (e.g., NP_006681), MMP25 (e.g., NP_071913), MMP26 (e.g., NP_068573), MMP2? (e.g., NP_071405), and/or MMP28 (e.g., NP_001027449). In some aspects, one or more of these may be excluded as an embodiment.

In certain embodiments, the matrix metalloproteinase is MMP1a. In certain embodiments, the macrophage is an M2-specific macrophage. In one embodiment, the recombinant targeting protein is a human integrin α1 encoded by SEQ ID NO: 3, the recombinant catalytic enzyme is a human MMP1 encoded by SEQ ID NO: 5, and wherein the macrophage is a human M2-specific macrophage. In certain aspects a macrophage can express a nucleic acid that is or is at least 70, 75, 80, 85, 90, 91, 92, 93, 94, 95, 96, 97, 98, 99, or 100% identical (or any range derivable therein) to SEQ ID NO:5 or SEQ ID NO:7, or a segment thereof. In other aspects a macrophage can express a polypeptide that is 80, 85, 90, 91, 92, 93, 94, 95, 96, 97, 98, 99, or 100% identical to SEQ ID NO:6 or SEQ ID NO:8, or a functional variant or segment thereof.

SEQ ID NO:5 provides the full length coding sequence of human MMP1 that encodes the amino acid sequence of SEQ ID NO:6. SEQ ID NO:6 is a 469 amino acid protein having a signal peptide from amino acid 1 to 17 (mature protein comprising amino acids 18 to 469 of SEQ ID NO:6) and a metalloprotease region from approximately amino acid 98 to 276 of SEQ ID NO:6. In certain aspects a segment of SEQ ID NO:6 can be expressed by an engineered macrophage, the segment comprising or comprising at least 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468 amino acids of SEQ ID NO:6 (or any range derivable therein) starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394 of SEQ ID NO:6 (or any range of positions therein). and ending at amino acid 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469 of SEQ ID NO:6 (or any range of positions therein).

SEQ ID NO:7 provides the full length coding sequence of mouse MMP1a that encodes the amino acid sequence of SEQ ID NO:8. SEQ ID NO:8 is a 464 amino acid protein having a signal peptide from amino acid 1 to 17 (mature protein comprising amino acids 18 to 464 of SEQ ID NO:8) and a metalloprotease region from approximately amino acid 95 to 274 of SEQ ID NO:8. In certain aspects a segment of SEQ ID NO:8 can be expressed by an engineered macrophage, the segment comprising or comprising at least 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468 amino acids of SEQ ID NO:8 (or any range derivable therein) starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389 of SEQ ID NO:8 (or any range of positions therein), and ending at amino acid 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464 of SEQ ID NO:8 (or any range of positions therein).

In a second aspect, a population of cells is contemplated that includes the genetically-engineered macrophage according to the first aspect and embodiments thereof.

In a third aspect, a cellular therapy product includes a genetically-engineered macrophage comprising at least one of a recombinant targeting protein and a recombinant catalytic enzyme. In one embodiment of the third aspect, the cellular therapy product further includes one or more cell media components and/or therapeutic compounds. In another embodiment of the third aspect, the cellular therapy product further includes an effective amount of one or more of α-tocopherol, interferon-γ, quercetin, an ACE inhibitor, and PPAR-δ. In another embodiment of the third aspect, the cellular therapy product further includes a pharmaceutical reagents and/or excipients suitable for therapeutic application.

The term “effective amount” means an amount effective, at dosages and for periods of time necessary, to achieve the desired therapeutic or prophylactic result. In regard to liver, cardiac, or lung fibrosis, an effective amount is a dose sufficient to prevent advancement, delay progression, or to cause regression of liver, cardiac, or lung fibrosis, or which is capable of reducing symptoms caused by the disease. In one example, an effective amount is an amount of a therapy sufficient to reduce inflammation in the liver, reduce liver enzyme levels (such as AST, ALT, and/or AP) and/or reduce scarring of the liver by at least 10%, at least 20%, at least 50%, at least 70%, or at least 90%. In one example, a effective amount is an amount of a therapy sufficient to increase liver, cardiac, or lung function in a fibrotic liver, heart, or lung, for example an increase of at least 10%, at least 20%, at least 50%, at least 70%, or at least 90% as compared to an absence of therapy. In certain aspects and effective amount of macrophages can include or include at least or at most 10, 100, 1000, 1×10⁴, 1×10⁵, 1×10⁶, 1×10⁷ 1×10⁸, 1×10⁹, 1×10¹⁰ macrophages, including all values and ranges there between.

In a fourth aspect, a method of treating an individual for fibrosis includes administering the cellular therapy product according to the third aspect and embodiments thereof. In one embodiment, the cellular therapy product is administered by injection to the individual (e.g., systemic or local injection). In certain aspects the injection is by intravenous injection. In other aspects the injection into or around (within 1 to 10 cm) of a fibrotic lesion or potential fibrotic area. When administering the cellular therapy product by injection, the administration may be by continuous infusion or by single or multiple boluses. In one embodiment, the cellular therapy product comprises of genetically-engineered macrophages derived from the individual being treated (i.e., autologous cells).

In a fifth aspect, a method of reversing or tretaing fibrosis in an individual in need thereof includes administering to the individual a genetically-engineered M2 macrophage capable of expressing recombinant ITGA-1 and MMP1 or MMP1a, targeting the macrophage to the fibrotic area of the individual, and reversing fibrosis within the targeted area.

In a sixth aspect, genetically engineered macrophages are made by transfecting M2-specific macrophages with one or more expression vector, e.g., lentiviral constructs, and selected for incorporation of the expression vector(s) and expression of the recombinant genes. Recombinant M2-specific recombinant macrophages expressing integrin A1, MMP1 or MMP1a, or both integrin A1 and MMP1 or MMP1a can be selected. The selected cell can be introduced into an individual as a novel therapeutic approach for liver, cardiac, lung fibrosis and other fibrotic diseases.

All publications, patents, and patent applications cited herein are hereby-expressly incorporated by reference in their entirety for all purposes.

As used herein, the singular forms “a,” “an,” and “the” include plural referents unless the context clearly dictates otherwise. For example, reference to “a metabolite” means one or more metabolites.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” as used herein represents the inherent degree of uncertainty that can be attributed to any quantitative comparison, value, measurement, or other representation. The term “substantially” is also used herein to represent the degree by which a quantitative representation can vary from a stated reference without resulting in a change in the basic function of the subject matter at issue.

Methods well known to those skilled in the art can be used to construct genetic expression constructs, targeting vectors, and genetically-engineered cells according to this invention. These methods include in vitro recombinant DNA techniques, synthetic techniques, in vivo recombination techniques, polymerase chain reaction (PCR) techniques, and others. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the term “genetically-engineered” refers to the genetic manipulation of one or more cells, whereby the genome of the one or more cells has been augmented by at least one DNA sequence. Candidate DNA sequences include but are not limited to genes that are not naturally present, DNA sequences that are not normally transcribed into RNA or translated into a protein (“expressed”), and other genes or DNA sequences which one desires to introduce into the one or more cells. It will be appreciated that typically the genome of genetically-engineered cells described herein is augmented through transient or stable introduction of one or more recombinant genes.

Generally, introduced DNA is not originally resident in the cell that is the recipient of the DNA, but it is within the scope of this disclosure to isolate a DNA segment from a given genetically-engineered cell, and to subsequently introduce one or more additional copies of that DNA into the same genetically-engineered cell, e.g., to enhance production of the product of a gene or alter the expression pattern of a gene. In some instances, the introduced DNA will modify or even replace an endogenous gene or DNA sequence by, e.g., homologous recombination, site-directed mutagenesis, and/or genome editing technology, including CRISPR (clustered regularly-interspaced short palindromic repeats), and/or mammalian transposon technology, such as by using the piggyBac™ transposon. In some instances, the introduced DNA is introduced into the recipient via viral vectors, including vectors derived from retrovirus, lentivirus, and adeno-associated virus. In some instances, the introduced DNA is introduced into the recipient cell directly with electroporation.

As used herein, the term “recombinant gene” refers to a gene or DNA sequence that is introduced into a genetically-engineered cell, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a cell by methods to form a genetically-engineered cell. It will be appreciated that a recombinant gene that is introduced into a cell can be identical to a DNA sequence that is normally present in the cell being transformed, and is introduced to provide one or more additional copies of the DNA to thereby permit overexpression or modified expression of the gene product of that DNA. Recombinant genes can also be introduced with different driving promoters or associated sequences that can alter the gene's expression level or pattern. Such recombinant genes are particularly-encoded by cDNA. Non-coding sequences, such as short hairpin RNAs, microRNAs, or long non-coding RNAs, may also be included.

It is further contemplated that recombinant genes can be codon optimized to maximize protein expression in genetically-engineered cells by increasing the translation efficiency of a particular gene. Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly-expressed genes. However, while optimal codon usage is contemplated herein for expression of disclosed proteins, all possible codons are contemplated for use herein for nucleic acids encoding any disclosed protein.

As used herein, the term “cellular therapy product” refers to a population of cells including one or more cells that has been genetically engineered to at least one of target a desired location within an individual and have a physiologically relevant effect at the desired location. For example, a cellular therapy product can be a population of cells including a genetically-engineered macrophage that can degrade collagen. The population of cells can be homogeneous (i.e., including only genetically-engineered macrophages) or heterogeneous (including genetically-engineered macrophages, non-genetically engineered macrophages, and other cell types whether genetically-engineered or not). A cellular therapy product can further include one or more cell media components (e.g., buffers, antibiotics, salts, vitamins, growth factors, amino acids, etc.) and/or therapeutic compounds to maintain the population of cells and/or treat a disease. For example, a cellular therapy product can include a genetically-engineered macrophage and an antibiotic. Cellular therapy products can further include additional therapeutic agents, such as one or more of α-tocopherol, interferon-γ, quercetin, an ACE inhibitor, and PPAR-δ. Additional therapeutic agents and pharmaceutical reagents and/or excipients suitable for therapeutic application can also be included in contemplated cellular therapy products. Additional reagents are contemplated for inclusion in cellular therapy products.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Is is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in to context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

These and other features and advantages of the present invention will be more fully understood from the following detailed description taken together with the accompanying claims. It is noted that the scope of the claims is defined by the recitations therein and not by the specific discussion of features and advantages set forth in the present description.

### DESCRIPTION OF DRAWINGS

FIGS. 1A and 1B illustrate the effectiveness of anti-inflammatory M2-specific macrophage treatment against CCl₄-mediated liver fibrosis in mice. FIG. 1A shows an ultrasound scan of a mouse liver after 10 weeks of CCl₄ treatment only. FIG. 1B shows an ultrasound scan of a mouse liver after 10 weeks of CCl₄ treatment followed by treatment with anti-inflammatory M2-specific macrophages, which can promote tissue repair and regeneration. Asterisks in each figure designate liver lobes, and signal intensity (brightness) indicates liver texture hardness, which correlates with fibrosis. The notable lesser intensity (brightness) in FIG. 1B compared to FIG. 1A indicates the effectiveness of the inventive anti-inflammatory M2-specific macrophage cell treatment in removing liver fibrosis.

FIGS. 2A and 2B show histochemical analyses of CCl₄-treated mouse livers. Treated mice were sacrificed and their livers removed, sectioned, and stained with hematoxylin and eosin. FIG. 2A shows a section of mouse liver after 10 weeks of CCl₄ treatment only. Inflammation, fibrotic lesions, and necrotic lesions are evident. FIG. 2B shows a section of mouse liver after 10 weeks of CCl₄ treatment followed by treatment with anti-inflammatory M2-specific macrophages. Marked reductions in inflammation and fibrotic lesions are evident in FIG. 2B compared to FIG. 2A (arrows). For each figure, the scale bar indicates 500 μm. Pathological evaluations are shown in Table No. 1.

FIGS. 3A and 3B show histochemical analyses of CCl₄-treated mouse livers. Treated mice were sacrificed and their livers removed, sectioned, and stained with trichrome staining for collagenous fibers. Arrows indicate areas of fibrotic lesions. FIG. 3A shows a section of mouse liver after 10 weeks of CCl₄ treatment only. Several areas of fibrosis are evident. FIG. 3B shows a section of mouse liver after 10 weeks of CCl₄ treatment followed by treatment with anti-inflammatory M2-specific macrophages. Marked reductions in fibrotic lesions are noted in FIG. 3B compared to FIG. 3A (arrows). For each figure, the scale bar indicates 500 μm.

FIGS. 4A and 4B show lentiviral constructs for the expression of integrin A1 (FIG. 4A) or MMP1 (FIG. 4B). Each vector encodes integrin or MMP1 driven by a CMV promoter and a selection marker (fluorescence protein tdTomato and puromycin resistant gene, Puro) driven by a constitutive promoter UbiC (Ubiquitin C promoter). TdTomato and Puro are separated by a self-cleavable peptide T2A.

FIGS. 5A and 5B show engraftment of engineered macrophages partially prevented MI-induced systolic dysfunction in left ventricle. There were marked deteriorations in Ejection Fraction (EF)(FIG. 5A) and Fraction Shortening (FS)(FIG. 5B) in MI mice receiving PBS injections, indicating an impaired systolic function/heart failure induced by LAD surgery. Ejection Fraction (EF) and Fraction Shorting (FS) in mice received engineered macrophage were higher than those received PBS, showing cardioprotective effect of engineered macrophage in post-MI heart.

FIGS. 6A and 6B show that cellular therapy using engineered macrophages prevented MI-induced LV dilation. Enlargement of LV chamber size was observed following surgical ligation of the LAD in PBS group. Engraftment of engineered macrophages prevented LV from MI-induced dilation. FIG. 6A LVID;d and FIG. 6B LVID;s.

FIGS. 7A and 7B show cellular therapy using engineered macrophages prevented ischemic myocardium remodeling. Myocardial infarction induced myocardium remodeling in PBS group, evidenced by an increase in heart weight. Lower heart weight in the engineered macrophages group indicates the cellular therapy regressed the remodeling progress. FIG. 7A HW/BW and FIG. 7B HW/T.

FIG. 8 shows cellular therapy using engineered macrophages prevented TAC-induced LV diastolic dysfunction. Increasing of E/A was observed following surgical constraining of the aorta in PBS group. Engraftment of engineered macrophages prevented LV from TAC-induced diastolic dysfunction.

FIGS. 9A, 9B, and 9C show the effect of Macrophage engraftment on BLM-induced lung injury in mice. H&E staining on tissue sections prepared from the lungs of C57BL6 mice 14 days after PBS/BLM exposure. (FIG. 9A): Control mice exposed to PBS and injected with PBS. (FIG. 9B): Mice in fibrosis group exposed to BLM then injected with PBS. (FIG. 9C). Macrophages treatment via tail vein injection reduced the fibrosis and the degree of inflammation in lungs of mice challenged with BLM.

### DETAILED DESCRIPTION

Embodiments described herein are directed to genetically-engineered macrophages capable of removing fibrotic scarring, for example, in liver, cardiac, or lung fibrosis. This disclosure is further directed to a cellular therapy product, such as an enriched population of genetically-engineered macrophages. Still further, this disclosure is directed to novel therapeutic approaches to enhance decomposition of fibrotic tissue and induce regeneration of functional hepatocytes by delivery of genetically-engineered macrophages to damaged liver. Additional characteristics and advantages of certain embodiments are described below.

### Cell Selection and Growth

Suitable cells that can be used in the present disclosure include, but are not limited to, macrophages. In one specific embodiment, contemplated cells for use herein include M2 macrophages that can turn off inflammatory responses and promote tissue wound repair, termed “anti-inflammatory M2-specific macrophages.”

In some embodiments, cells can be taken from an individual (autologous source) to be treated, genetically-modified, and introduced (e.g., by injection) back into the individual to remove fibrotic scars in the individual's liver, heart, lung, or other tissue or organ. In one embodiment, such a cellular therapy product can be derived from an apheresis product taken from the individual. In another embodiment, a cellular therapy product intended for an individual can be derived from an apheresis product taken from another individual (heterologous source) or from another cell source. In one embodiment, a suitable autologous macrophage population can be produced as described in Fraser et al. (Development, functional characterization and validation of methodology for GMP-compliant manufacture of phagocytic macrophages: A novel cellular therapeutic for liver cirrhosis. Cytotherapy 2017 September; 19(9):1113-1124).

The methods for the treatment of fibrosis in a human or other mammalian subject by administering engineered M2 macrophages to the subject at the site of fibrosis. The source of macrophages can be peripheral blood or tissue at or near the site of inflammation. The source of macrophages may be an isolated source, which comprises an ex-vivo composition comprising macrophages. Such a composition may be a culture of macrophages, a macrophage-containing tissue obtained from a subject (which may be the subject to be treated), or a culture, such as a culture comprising monocytes.

The source of macrophages may be a concentrated macrophage solution generated by fractionating peripheral blood obtained from the patient. Fractionating peripheral blood comprises preparing a suspension of peripheral blood mononuclear cells (PBMCs) and inducing the PBMCs to differentiate into macrophages. Preparing a suspension of PBMCs from peripheral blood can be performed by any method commonly known in the art. As a non-limiting example, PBMCs can be prepared by Ficoll gradient centrifugation. Ficoll gradient centrifugation includes transferring a volume of Ficoll in a tube, such as a test tube. Whole blood is then gently overlayed onto the Ficoll and the tube is centrifuged for from about 15 minutes to about 60 minutes at from about 175 g to about 225 g at room temperature. In a preferred embodiment, the tube is centrifuged for 45 minutes at 200 g. After centrifugation, there remains a pellet of red blood cells, a Ficoll layer, a white layer comprising PBMCs, and a plasma layer. The white layer comprising PBMCs can then be removed from the tube. Because the PBMCs include monocytes and lymphocytes, the PBMCs can be processed to isolate the monocytes. For example, an Anti-CX3CR1 MicroBeads Kit (Miltenyi Biotec Inc., Auburn, Calif.) can be used to specifically bind monocytes to magnetic beads, which can then be separated from the lymphocytes. Alternatively, the PBMCs can be separated from lymphocytes by flow cytometry techniques, such as fluorescence-activated cell sorting (FACS). After isolation, PBMCs can be cultured in Macrophage Base Medium DXF (PrmoCell), which does not induce differentiation. Differentiation of PBMCs or isolated monocytes into macrophages can be induced by culturing the PBMCs or isolated monocytes, for example, in the presence of differentiation medium containing macrophage colony-stimulating factor (M-CSF) or granulocyte-macrophage colony-stimulating factor (GM-CSF). In various embodiments, a differentiation medium is Macrophage Base Medium DXF (Promocell, Heidelberg, Germany). Once differentiated into macrophages, the macrophages can be suspended in a medium to generate the concentrated macrophage solution. The M2 macrophages can then be manipulated, e.g., transfected and engineered, to produce the targeted macrophages described herein.

Culturing Process. The culture medium to be used may be a basic culture medium containing components (inorganic salts, carbohydrates, hormones, essential amino acids, non-essential amino acids, and vitamins) and the like required for the cell's viable growth. Examples of the culture medium include Dulbecco's Modified Eagle's Medium (DMEM), Minimum Essential Medium (MEM), Basal Medium Eagle (BME), Dulbecco's Modified Eagle's Medium: Nutrient Mixture F-12 (DMEM/F-12), Glasgow Minimum Essential Medium (Glasgow MEM), Gibco® RPMI 1640 culture medium (manufactured by Life Technologies), HL-1 known composition, serum-free culture medium (manufactured by Lonza Inc.), and the like. In the culturing process, the culture medium may be suitably replaced with a new one according to the growth rate of the cells.

In addition, a compound inducing the differentiation or trait of the macrophage may be added to the culture medium to be used. By adding the compound, the rate of differentiation or trait change can be further accelerated, and differentiation or trait can be controlled in a certain direction. Examples of compounds that trait-induce the macrophage into the M1 macrophage include Th1 cytokines such as interferon (IFN)-γ, tumor necrosis factor (TNF)-α, lipopolysaccharide (LPS) and the like, and two or more of these compounds may be used in combination. In addition, examples of compounds that trait-induce the macrophage into the M2 macrophage include Th2 cytokines such as interleukin (IL)-4 and IL-13, and two or more of these compounds may be used in combination. In addition, the compounds trait-inducing into the M1 macrophage and the compounds trait-inducing into the M2 macrophage may be used in combination.

The concentration of the compounds that induce the macrophage differentiation is not particularly limited, and may be 1 nM or more and 1 μM or less, and may be 5 nM or more and 100 nM or less. Within the above range, it is possible to more efficiently induce the trait from the macrophage into the M1 or M2 macrophage.

Culture conditions are not particularly limited as long as it is a method suitable for culturing the macrophage, for example, the density of seeding the macrophage in the culture medium is preferably 1×10⁰ to 1×10⁷ cells/mL, and more preferably 1×10² to 1×10⁶ cells/mL. The culture temperature is preferably 25° C. or more and 40° C. or less, more preferably 30° C. or more and 39° C. or less, and further preferably 35° C. or more and 39° C. or less. The culturing time can be appropriately set depending on the growth state of the macrophage, and it is preferably 1 hour or more and 100 hours or less. The culture environment is preferably cultured under CO₂ conditions through approximately 5% carbon dioxide.

### Genetic Constructs

In some embodiments, genetically-engineered macrophages of the present invention can include one or more recombinant genes. Genetic constructs contemplated for use herein can be transiently expressed or permanently expressed in a recombinant host cell. In one particular embodiment, a genetically-engineered macrophage can include one or more genes that can be used to target the cell (e.g., a macrophage) to a desired location, such as the liver, heart, lung or specifically to a fibrotic scar. For example, a genetically-engineered macrophage can include one or more recombinant collagen receptors or subunits thereof. Examples of contemplated collagen receptors useful herein include, but are not limited to, integrins. In one embodiment, genetically-engineered macrophages include one or more of subunits of α1β1, α2β1, α10β1, and/or α11β1 integrins. Specific examples include integrin A1 or α1 (ITGA-1), such as shown in SEQ ID NO:1, SEQ ID NO:2, SEQ ID NO:3, and/or SEQ ID NO:4. Other contemplated collagen receptors include discoidin domain receptors, such as DDR1 (e.g., NP_001189450) and/or DDR2 (e.g., NP_001014796), mannose family receptors, such as M-phospholipase A2 receptor (e.g., NP_001007268 and Endo180 receptor (e.g., P22897), and immunoglobulin-like receptors, such as glycoprotein VI (e.g. NP_001077368). In one particular embodiment, a genetically-engineered macrophage includes and expresses ITGA-1 (integrin a subunit 1). While not wishing to be bound by theory, it is believed that expression of one or more targeting proteins, such as a collagen receptor or subunit thereof, will not only augment targeting of genetically-engineered macrophages to the liver, heart, lung or other tissue, but will also cause the macrophages to be retained at the site of damage (a collagen-rich environment) for a longer period of time and thereby increase their efficacy, specificity, and safety for treating fibrosis.

In another embodiment, a genetically-engineered macrophage of the present invention can include one or more genes that enhance fibrosis (e.g., liver, cardiac, or lung) degradation. For example, a genetically-engineered macrophage of the present invention can include one or more collagenases. In one particular example contemplated herein, genetically-engineered macrophages described herein include and express one or more matrix metalloproteinases (MMPs). Examples of contemplated MMPs include, but are not limited to, MMP1, MMP1a, MMP2, MMP3, MMP7, MMP8, MMP9, MMP10, MMP12, MMP13, MMP14, MMP17, MMP19, MMP20, MMP21, MMP22, MMP24, MMP25, MMP26, MMP27, and MMP28 (Caley et al. Adv. Wound Care (New Rochelle) 2015, 4:225-34). In some embodiments, one or more MMPs may be excluded.

It is further contemplated that genetically-engineered macrophages of the present invention can include other organ or tissue-specific targeting proteins, peptides, and/or molecules and/or other catalytic enzymes or substances to remove fibrotic scars from an afflicted individual.

### Treatment Methodologies

In some embodiments of the present invention, methods of treating an individual for fibrosis are contemplated. Examples of conditions that can be treated include liver fibrosis, cardiac fibrosis, pulmonary fibrosis, arthrofibrosis, myelofibrosis, mediastinal fibrosis, retroperitoneal fibrosis, nephrogenic systemic fibrosis, as well as keloids, Crohn's disease, fibrocystic breasts, and Peyronie' s disease, among others. In one specific embodiment, a method of treating an individual for liver, cardiac, or lung fibrosis includes acquiring a population of macrophages, genetically-engineering the population of macrophages to express a fibrosis targeting protein, and administering the population of genetically-engineered macrophages to the individual.

Genetically-engineered macrophages of the present invention can be prepared and used immediately to treat an individual in need thereof. Alternatively, a population of genetically-engineered macrophages can be prepared and frozen for later use.

Administration of genetically-engineered macrophages can be through any means generally accepted for the administration of cells to an individual (e.g., intravenously). In some embodiments, genetically-engineered macrophages can be introduced into an individual in need thereof by portal vein injection, intracardiac injection, or intravenous (IV) injection.

Liver fibrosis. Liver fibrosis or fibrotic scarring of the liver often occurs in patients with chronic liver disease. Diseases such as hepatitis infection (via hepatitis B virus or hepatitis C virus), Wilson's disease, blocked bile duct, non-alchoholic fatty liver and alcohol abuse (such as alcohol use disorder or “AUD”) commonly lead to the development of liver fibrosis, though exposure to toxins and trauma have also been associated with the condition. Liver fibrosis is the result of excessive accumulation of extracellular matrix (ECM) proteins, especially α1 collagen, produced by cells such as hepatic stellate cells (HSCs) responding to liver injury (i.e., chronic activation of the wound-healing reaction).

Typically, at least several months to years of ongoing liver injury are required to cause fibrosis. Advanced liver fibrosis can lead to cirrhosis, hepatic insufficiency, portal hypertension, and liver failure. There are few treatment options for patients with end-stage chronic liver disease with liver transplantation being the last resort for those whose liver has been damaged beyond its capacity to regenerate. However, liver transplantation is an extremely invasive and risky medical intervention. As well, patients with end-stage liver disease are often not eligible for transplantation. Moreover, liver transplantation is extremely-expensive, and can cost in excess of $600,000 in the United States. Therefore, new treatment options are needed for individuals with liver fibrosis.

One potential treatment option is to reverse liver fibrosis. Approaches to reversing liver fibrosis have been under investigation for nearly 50 years. Even so, the best line of attack for reversing liver fibrosis remains to be attempting to remove the primary disease causing the fibrosis and allowing the liver to regenerate. Even so, liver regeneration cannot always fully reverse liver fibrosis, and the ability of the liver to regenerate is progressively lost in individuals with advancing liver disease. Therefore, ongoing therapeutic investigations are developing an antifibrotic armamentarium of chemical compounds aimed at various molecular and cellular targets to prevent or slow fibrosis. Examples of antifibrotic chemical candidates include α-tocopherol (inhibits HSC activation), interferon-γ (inhibits ECM synthesis in HSCs), quercetin (antioxidant), ACE inhibitors (inhibit HSC proliferation), and PPAR-δ (see Houlum et al. Gastroenterology 1997, 113:1069-73; Rockey-et al. J Investig Med. 1994, 42:660-70; Pavanato et al. Dig Dis Sci. 2003, 48:824-9; Warner et al. Clin Sci (Loud) 2007, 113:109-18; Marra et al. Gastroenterology 2000, 119:466-78). However, many of these nascent therapeutic candidates apparently function by preventing development of liver fibrosis (inhibiting chronic wound healing) rather than by removing existing fibrotic scarring. There are alternative approaches for reversing liver fibrosis.

One alternative approach for treating liver disease is being explored that utilizes bone marrow cell therapy for improving liver fibrosis. Using animal models of experimental liver damage has shown that macrophages can play a key role in the control and repair of fibrotic liver disease (Ramachandran et al. Proc Natl Acad Sci USA 2012, 109: E3186-95). Indeed, some studies of bone marrow cell therapy for liver cirrhosis have shown improvements in several clinical parameters in experimental chronic liver injury. (Thomas et al. Hepatology 2011, June; 53(6):2003-15). However, existing cell-based approaches have limited efficacy.

Cardiac fibrosis. Cardiac fibrosis, a hallmark of heart disease, is thought to contribute to sudden cardiac death, ventricular tachyarrhythmia, left ventricular (LV) dysfunction, and heart failure. Cardiac fibrosis is characterized by a disproportionate accumulation of fibrillated collagen that occurs after myocyte death, inflammation, enhanced workload, hypertrophy, and stimulation by a number of hormones, cytokines, and growth factors.

Cardiac fibrosis may also refer to an abnormal thickening of the heart valves due to inappropriate proliferation of cardiac fibroblasts but more commonly refers to the proliferation of fibroblasts in the cardiac muscle. Fibrocyte cells normally secrete collagen, and function to provide structural support for the heart. When over-activated this process causes thickening and fibrosis of the valve, with white tissue building up primarily on the tricuspid valve, but also occurring on the pulmonary valve. The thickening and loss of flexibility eventually may lead to valvular dysfunction and right-sided heart failure.

The most obvious treatment for cardiac valve fibrosis or fibrosis in other locations, consists of stopping the stimulatory drug or production of serotonin. Surgical tricuspid valve replacement for severe stenosis (blockage of blood flow) has been necessary in some patients. Also, a compound found in red wine, resveratrol, has been found to slow the development of cardiac fibrosis. (Olson et al. (2005) American journal of physiology. Heart and circulatory physiology 288(3):H1131-8; Aubin, et al. (2008) The Journal of Pharmacology and Experimental Therapeutics 325(3):961-8). More sophisticated approaches of countering cardiac fibrosis like microRNA inhibition (miR-21, for example) are being tested in animal models.

Heart disease is the major cause of mortality in developed countries, accounting for an annual death of about 800,000 in United States alone. Numerous forms of cardiovascular disease exist that have differential pathological observations. Most cardiac diseases are associated with cardiac fibrosis that refers to an abnormal scarring process of heart valves caused by inappropriate proliferation of myofibroblast and excessive deposition of extracellular matrix (ECM) proteins in cardiac muscle. Myofibroblasts are principally responsible for deposition of the excessive fibrotic ECM. (Travers et al. Circ Res, 2016, 118(6):1021-40).

Activation of cardiac fibrosis has been extensively studied in the past few decades. In response to acute cardiac injury like ischemia or myocardium infarction, or chronic disease like hypertension, diabetic cardiomyopathy, Cardiac Fibroblast (CFs) within the connective tissue in the heart is activated and transformed to myofibroblasts, which induce excessive extracellular matrix (ECM) deposition.(Liu et al. Front Physiol., 2017, 8:238; Tian et al. Exp Ther Med 2017, 13(5):1660-4).

There are two most common types of cardiac fibrosis, Reactive Interstitial Fibrosis (RIF) and Replacement Fibrosis (RF). RIF is often induced by one or multiple progressive chronic courses (e.g., diabetics and hypertension) that is characterized by diffused deposition of collagen protein (a type of ECM) and increased interstitial compartment volume. RF occurs after acute injury while the expansion of ECM and elevated collagen I deposition replace the dead cardiomyocyte in order to prevent the infarcted myocardium from rupture. In general, the increased cardiac fibrosis leads to distorted organ architecture and function that results in heart failure. (McLenachan and Dargie, Am J Hypertens 1990, 3(10):735-40; Krenning et al. J Cell Physiol 2010, 225(3):631-7; Mewton et al., J Am Coll Cardiol 2011, 57(8):891-903).

During the pathological process of cardiac fibrosis, the necrotic and apoptotic cardiomyocytes trigger the excessive accumulation of ECM proteins in both RIF and RF. Thus it is hypothesized that, using macrophage subsets with anti-inflammatory properties may have direct anti-fibrotic effects by clearing necrotic and apoptotic cells and suppressing fibroblast activation. In this studies described below, macrophages were injected directly into ischemic mouse heart and monitored the cardiac function to investigate the anti-fibrotic potential of the cellular therapy.

Current clinical therapies for cardiac fibrosis mainly rely on established pharmacological agents. ACE inhibitors, statins and aldosterone antagonists are among the drugs that have been shown to exert beneficial effects on cardiac fibrosis. ACE inhibitors like Lisinopril regress cardiac fibrosis and improve LV function in patients with hypertension. Statins treatment with Atorvastatin reduces fibrotic biomarker in heart failure patients. Spironolactone, an aldosterone antagonist, can reduce cardiac fibrosis in cardiomyopathy. Nevertheless, existing treatments have several major shortcomings: (1)These drugs can only moderately improve the heart functions; (2)What is more problematic is none of the existing therapies exclusively treats fibrosis in the heart; (3) these treatments target the causes or symptoms but fail to effectively inhibit myocardial scar formation, which leaves the patients with severe cardiac fibrosis with little options. New compounds targeting key components of pro-fibrotic pathways are being tested on animal models and pre-clinical trials, but so far the results are mixed and clinical translations are very limited. Lack of effective clinical treatment for cardiac fibrosis brings an urgent need for developing novel, tissue-specific and effective therapeutic approaches using unconventional strategy like cellular therapy with engineered macrophages.

The methods described herein are suitable for treating an individual who has been diagnosed with a disease related to progressive cardiac fibrosis, who is suspected of having a disease related to progressive cardiac fibrosis, who is known to be susceptible and who is considered likely to develop a disease related to progressive cardiac fibrosis, or who is considered likely to develop a recurrence of a previously treated disease relating to progressive cardiac fibrosis.

Existing evidence demonstrates the association of fibrosis with the heart failure process in a variety of heart diseases, including those associated with both volume and pressure overload (Maron et al, Am. J. Cardiol., 35:725-39 (1975); Schwarz et al, Am. J. Cardiol., 42:661-69 (1978); Fuster et al, Circ., 55:504-08 (1976); Bartosova et al, J. Physiol., 200:285-95 (1969); Weber et al, Circ., 83:1849-65 (1991); Schaper et al, Basic Res. Cardiol., 87:S1303-S1309 (1992); Boluyt et al, Circ. Res., 75:23-32 (1994); and Bishop et al, J. Mol. Cell Cardiol., 22:1157-65 (1990)). In the setting of heart failure, fibrosis involves an increase in both fibroblast number and matrix deposition (Morkin et al, Am. J. Physiol., 215:1409-13 (1968); Skosey et al, Circ. Res., 31:145-57 (1972); and Booz et al, Cardiovasc. Res., 30:537-43 (1995)), suggesting the importance of the fibroblast in the development of this condition. Cardiac fibroblasts are also the predominant source of synthesis of interstitial proteins and other myocardial components which have been implicated in heart failure by their effects on diastolic function and, indirectly, by effects on cardiac myocytes to cause or potentiate systolic dysfunction (Hess et al, Circ., 63:360-71 (1981); Villari et al, Am J. Cardiol., 69:927-34 (1992); Villari et al, JACC, 22:1477-84 (1993); Brilla et al, Circ. Res., 69:107-15 (1991); and Sabbah et al, Mol. & Cell Biochem., 147:29-34 (1995)).

The treatment of the fibrotic cardiac disease state can be determined by measuring one or more diagnostic parameters indicative of the course of the disease, compared to a suitable control. For comparison with animal models, a “suitable control” is an animal not treated with relaxin, or treated with the pharmaceutical formulation without relaxin. In the case of a human subject, a “suitable control” may be the individual before treatment, or may be a human (e.g., an age-matched or similar control) treated with a placebo.

Cardiac fibrosis to be treated by the methods of the present invention may be due to a variety of diseases associated with cardiac fibroblast proliferation or the activation of extracellular matrix protein synthesis by cardiac fibroblasts. These diseases may be effectively treated in the present invention. Such diseases include aortic and mitral valvular regurgitation. In addition, cardiac hypertrophy, which is associated with many cardiac diseases, and often involves myocyte and fibroblast components, may be effectively treated in the present invention.

Heart failure is defined as the inability of the cardiac pump to move blood as needed to provide for the metabolic needs of body tissue. Decreases in pumping ability arise most often from loss or damage of myocardial tissue. As a result, ventricular emptying is suppressed which leads to an increase in ventricular filling pressure and ventricular wall stress, and to a decrease in cardiac output. As a physiological response to the decrease in cardiac output, numerous neuroendocrine reflexes are activated which cause systemic vasoconstriction, sympathetic stimulation of the heart and fluid retention. Although these reflex responses tend to enhance cardiac output initially, they are detrimental in the long term. The resulting increases in peripheral resistance increase the afterload on the heart and the increases in blood volume further increase ventricular filling pressure. These changes, together with the increased sympathetic stimulation of the heart, lead to further and often decompensating demands on the remaining functional myocardium.

Congestive heart failure, which is a common end point for many cardiovascular disorders, results when the heart is unable to adequately perfuse the peripheral tissues. According to recent estimates, there are about 4 million people in the United States diagnosed with this disease, and more than 50% of these cases are fatal within 5 years of diagnosis (Taylor et al., Annual Reports in Med. Chem. 22, 85-94 (1987)).

Lung fibrosis and Pulmonary Fibrosis Diseases. Pulmonary fibrosis disease is a devastating chronic lung disease resulting in scarring (fibrosis) of the lungs. Over time, the scarring gets worse and it becomes hard to take in a deep breath and the lungs cannot take in enough oxygen. Lung function decline is gradual, with the potential for intermittent, unpredictable, acute exacerbations and the development of associated pulmonary hypertension. Sometimes doctors can identify the cause of the fibrosis, but in most cases, they cannot. They call these cases idiopathic pulmonary fibrosis (IPF).

Pulmonary fibrosis disease primarily affect middle aged and older adults. About 50,000 people in the U.S. have idiopathic pulmonary fibrosis and an estimated 15,000 new cases develop each year. According to NIH/National Heart Lung, and Blood Institute, currently, no medicines are proven to slow the progression of IPF. Prednisone, azathioprine and N-acetylcysteine have been used to treat IPF, either alone or in combination. However, experts have not found enough evidence to support their use.

### Cotherapies

In some embodiments, cotherapies are envisioned in the present application. For example, a method of treating an individual with liver fibrosis can include introducing a cellular therapy product including a genetically-engineered macrophage into the individual and administering to the individual an effective amount of one or more of α-tocopherol, interferon-γ, quercetin, an ACE inhibitor, and PPAR-δ.

In certain instances method of treating may further involve performing surgery on the patient, such as by resecting all or part of the liver or fibrotic regions of the liver. Cellular therapy product may be administered to the patient before, after, and/or at the same time as surgery. In certain aspects the methods can be used to ameliorate fibrosis resulting from surgery and assist in regeneration. In other aspects, the methods can be used treat or reducing fibrotic areas not removed by surgery.

### Polypeptide Composition

“Polypeptide” refers to any peptide or protein comprising amino acids joined by peptide bonds or modified peptide bonds. “Polypeptide” can include short chain polypeptides, including peptides, oligopeptides or oligomers, and longer chain polypeptides, including proteins. Polypeptides may contain amino acids other than the 20 gene-encoded amino acids. “Polypeptides” include amino acid sequences modified either by natural processes, such as post-translational processing, or by chemical modification or other synthetic techniques well known in the art. Modifications can occur anywhere in a polypeptide, including the peptide backbone, the amino acid side-chains, and the amino terminus or the carboxy terminus. It will be appreciated that the same type of modification may be present in the same or varying degrees at several sites in a given polypeptide. Also, a given polypeptide may contain many types of modifications. Modifications include terminal fusion (N- and/or C-terminal), acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated polypeptide refers to one that can be administered to a subject as an isolated polypeptide; in other words, the polypeptide may not simply be considered “isolated” if it is adhered to a column or embedded in a gel. Moreover, an “isolated nucleic acid fragment” or “isolated peptide” is a nucleic acid or protein fragment that is not naturally occurring as a fragment and/or is not typically in the functional state.

The term “amino acid” or “residue” should be understood to mean a compound containing an amino group (NH₂), a carboxylic acid group (COOH), and any of various side groups, that have the basic formula NH₂CHRCOOH, and that link together by peptide bonds to form proteins. Amino acids may, for example, be acidic, basic, aromatic, polar or derivatized. Non-standard amino acids may be referred to as “non-canonical” amino acids. Amino acids are naturally found in the α- and L-form, however, β- and D-form amino acids can also be prepared.

A one-letter abbreviation system is frequently applied to designate the identities of the twenty “canonical” amino acid residues generally incorporated into naturally occurring peptides and proteins, these designation are well known in the art. Such one-letter abbreviations are entirely interchangeable in meaning with three-letter abbreviations, or non-abbreviated amino acid names. The canonical amino acids and their three letter and one letter codes include Alanine (Ala) A, Glutamine (Gln) Q, Leucine (Leu) L, Serine (Ser) S, Arginine (Arg) R, Glutamic Acid (Glu) E, Lysine (Lys) K, Threonine (Thr) T, Asparagine (Asn) N, Glycine (Gly) G, Methionine (Met) M, Tryptophan (Trp) W, Aspartic Acid (Asp) D, Histidine (His) H, Phenylalanine (Phe) F, Tyrosine (Tyr) Y, Cysteine (Cys) C, Isoleucine (Ile) I, Proline (Pro) P, and Valine (Val) V.

Certain embodiments also include variants of the polypeptides described herein. Variants of the disclosed polypeptides may be generated by making amino acid additions or insertions, amino acid deletions, amino acid substitutions, and/or chemical derivatives of amino acid residues within the polypeptide sequence. Desired amino acid substitutions (whether conservative or non-conservative) can be determined by those skilled in the art in accordance with guidance provided herein for increasing stability, while maintaining or enhancing potency of the polypeptides. In certain embodiments, conservative amino acid substitutions can encompass non-naturally occurring amino acid residues which are typically incorporated by chemical peptide synthesis rather than by synthesis in biological systems.

Conservative modifications can produce peptides having functional, physical, and chemical characteristics similar to those of the peptide from which such modifications are made. In contrast, substantial modifications in the functional and/or chemical characteristics of peptides may be accomplished by selecting substitutions in the amino acid sequence that differ significantly in their effect on maintaining (a) the structure of the molecular backbone in the region of the substitution, for example, as an α-helical conformation, (b) the charge or hydrophobicity of the molecule at the target site, or (c) the size of the molecule. For example, a “conservative amino acid substitution” may involve a substitution of a native amino acid residue with a non-native residue such that there is little or no effect on the polarity or charge of the amino acid residue at that position.

Recombinant DNA- and/or RNA-mediated protein expression and protein engineering techniques, or any other methods of preparing peptides, are applicable to the making of the polypeptides disclosed herein or expressing the polypeptides disclosed herein in a target cell or tissue. The term “recombinant” should be understood to mean that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. Examples of such molecular biological procedures are found in Maniatis et al., Molecular Cloning. A Laboratory Manual, Cold Spring Harbor Laboratory, Cold Spring Harbor, N.Y., 1982. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The polypeptides can be made in transformed host cells according to methods known to those of skill in the art. Briefly, a recombinant DNA molecule, or construct, coding for the peptide is prepared. Methods of preparing such DNA molecules are well known in the art. For instance, sequences encoding the peptides can be excised from DNA using suitable restriction enzymes. Any of a large number of available and well-known host cells may be used in the practice of various embodiments. The selection of a particular host is dependent upon a number of factors, which include, for example, compatibility with the chosen expression vector, toxicity of the polypeptides encoded by the DNA molecule, rate of transformation, ease of recovery of the polypeptides, expression characteristics, bio-safety, and costs. A balance of these factors should be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) cells, e.g., CHO cells and HEK293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired polypeptides are expressed. In addition, the DNA optionally further encode, 5′ to the coding region of a fusion protein, a signal peptide sequence (e.g., a secretory signal peptide) operably linked to the expressed polypeptide.

### Expression and Expression Vectors

The nucleic acids encoding any polypeptide(s) described herein can be inserted into or employed with any suitable expression system. Recombinant expression can be accomplished using a vector, such as a plasmid, virus, etc. The vector can include a promoter operably linked to nucleic acid encoding one or more polypeptides. The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing nucleic acids encoding proteases can be produced. Such expression vectors include, for example, pET, pET3d, pCR2.1, pBAD, pUC, and yeast vectors. The vectors can be used, for example, in a variety of in vivo and in vitro situations. The vector may be a gene therapy vector, for example an adenovirus vector, a lentivirus vector or a CRISP-R vector.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid to be expressed, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that are linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding a protease. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 nucleotides in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the expression constructs.

The expression of one or more protease from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pClneo-CMV.

The expression cassette or vector can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern and Berg, Molec. Appl. Genet. 1: 327 (1982)), mycophenolic acid, (Mulligan and Berg, Science 209: 1422 (1980)) or hygromycin, (Sugden et al., Mol. Cell. Biol. 5: 410-13 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes or viruses. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff et al., Science, 247, 1465-1468, (1990); and Wolff, Nature, 352, 815-818, (1991).

For example, the nucleic acid molecule, expression cassette and/or vector encoding a protease can be introduced to a cell by any method including, but not limited to, calcium-mediated transformation, electroporation, microinjection, lipofection, particle bombardment and the like. The cells can be expanded in culture and then administered to a subject, e.g., a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

### EXAMPLES

The Examples that follow are illustrative of specific embodiments of the invention, and various uses thereof. They are set forth for explanatory purposes only and are not taken as limiting the invention.

### Example 1

### Reduction of Liver Fibrosis in an Animal Model of Cirrhosis

In this example, M2-specific macrophages were used to treat an animal model of cirrhosis to demonstrate the ability of the macrophages to reverse liver fibrosis.

Animals. Six to eight week old male C57BJ/6 mice were purchased from the Jackson Lab and housed under specific pathogen-free conditions in the University of Chicago animal core facility. Animals consumed a standard sterile diet and filtered water ad libitum under a 12 hr light-dark cycle. The experimental protocol was approved by the Animal Care and Use Committee and the Ethics Committee of University of Chicago.

Induction of cirrhosis. Mice were intraperitoneally injected with 20% CCl₄ in corn oil at a dose of 0.1 mL/10 g body weight for 6-8 weeks to induce cirrhosis.

Treatment. Sedated mice were placed in a supine position with abdomen exposed and disinfected. Buprenorphine was subcutaneously given at a dose of 0.1 mg/kg before surgery. After a single 1.5 cm incision was made along the middle line by starting below the diaphragm, surgically exposing the portal vein without damaging intestines, liver, or diaphragm, 3.0×10⁶ M2-specific macrophages were collected in 100 μL PBS and slowly injected into the portal vein towards the liver mass. Incisions were closed using Nylon sutures. One hundred microliters of bupivacaine (5 mg/mL) were injected along the incision site for local pain management. One half milliliter of sterile saline was injected subcutaneously for hydration. Buprenorphine was re-administered every 12 hours for up to 72 hrs.

Ultrasonic Scan. At week 10 post CCl₄ treatment, sedated mice were placed in a supine position with abdomen hair removed. Due to the disproportional ratio of the ultrasonic probe to mouse body size, only longitudinal scans from the outer margin of the left side lobe to the outer margin of the far right side lobe were conducted for images of liver tissue texture reflection. Fibrotic tissues gave relatively stronger echo signals.

Histology. Subsequent to ultrasonic liver scans of the treated mice, liver lobes were collected and fixed in 10% formalin for histology. Trichrome and hematoxylin and eosin staining were performed on the fixed liver tissue samples. Hematoxylin and eosin staining was for general pathological evaluation and trichrome staining highlights collagen fibers. Histological evaluation for each group was performed by following HAI-Knodell Score system, one of the most recognized numeric scoring systems for pathologists to evaluate acute and chronic hepatic conditions in terms of liver parenchymal damage, inflammation, and fibrotic lesions. As shown in Table 1, all the listed aspects of hepatic pathological appearance were examined and scored with various weights. The total scores of each sample indicate the severity of liver damage and the efficacy of the treatment in a semi-quantitative way.

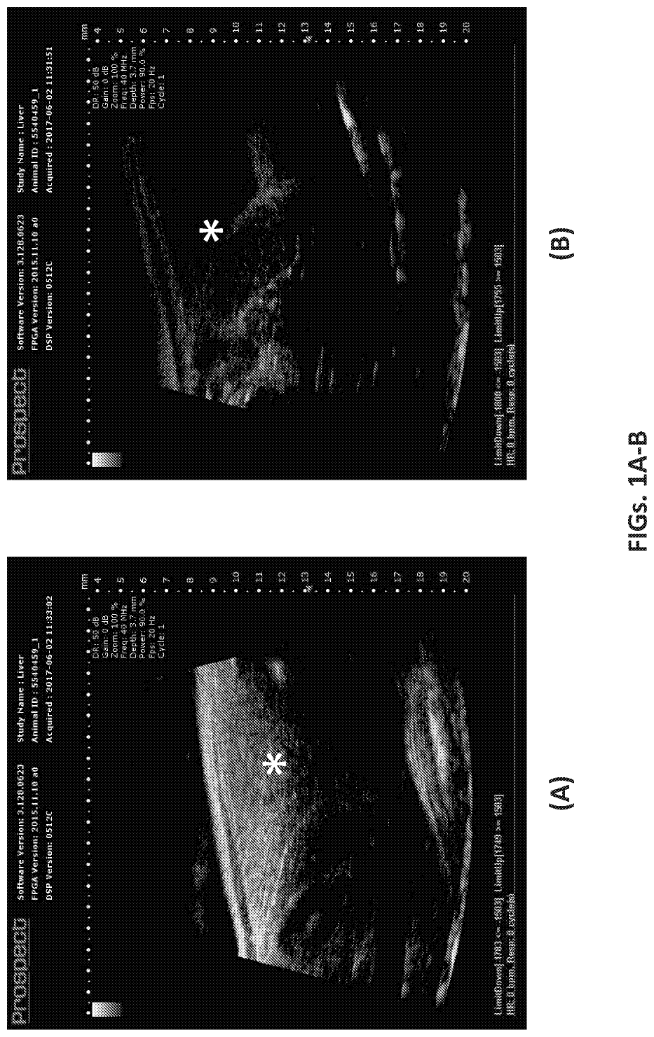

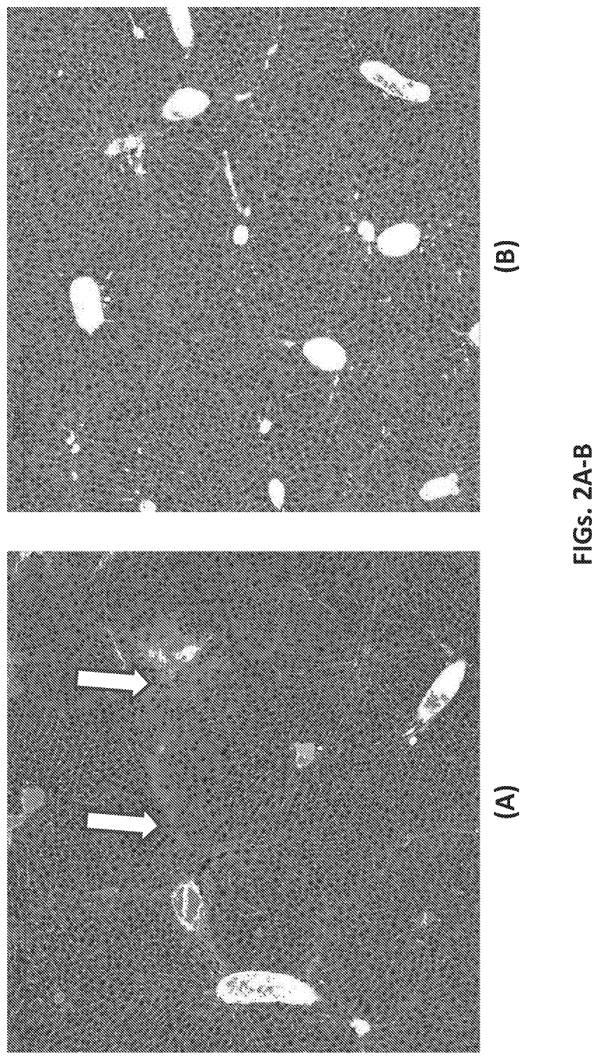

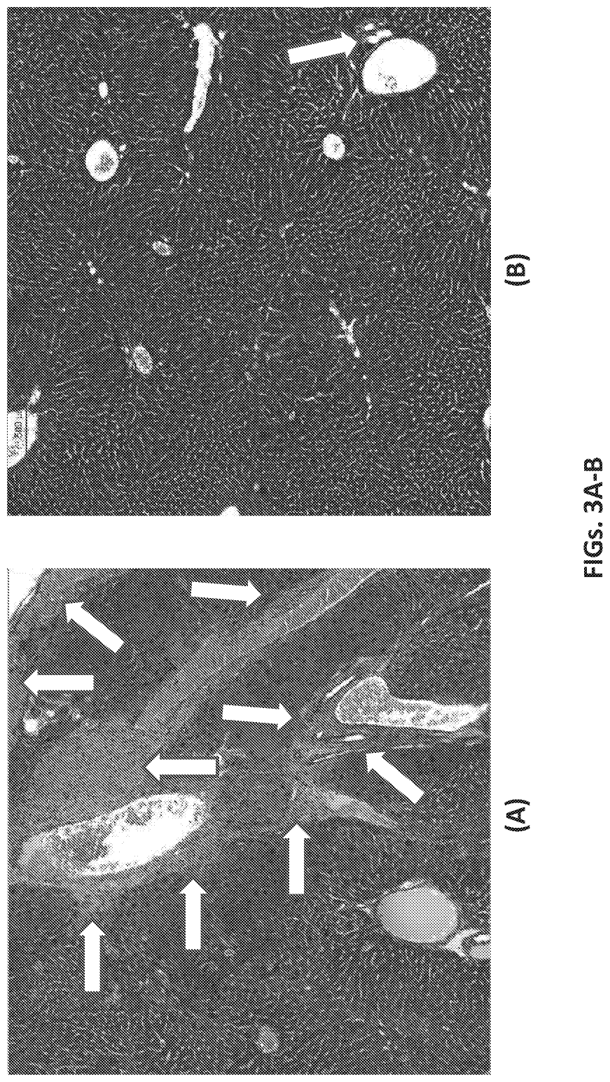

The results from this study demonstrate that the M2-specific macrophages significantly reversed established liver fibrosis in a mouse model of cirrhosis. As can be seen in FIG. 1, a marked reduction in liver fibrosis is evident based on ultrasonic scans. Treatment of cirrhotic mice with M2-specific macrophages led to marked reductions in inflammation and fibrotic lesions (see FIG. 2B compared to FIG. 2A). The reversal of liver fibrosis is further highlighted by the considerable reduction in number and size of liver fibrotic lesions in treated mice shown in FIG. 3B.

Further, a histology index was employed based on a previously reported index (see Knodell RG, et al. Hepatology 1981,1(5):431-5). Results of the histological assessment shown in Table 1 strongly suggest that portal vein delivery of macrophages significantly reverses liver damage by reducing fibrosis.

TABLE 1
Histology Index (HAI-Knodell Score)

| | | | | Treated | Treated |
|---|---|---|---|---|---|
| | Score | Ctrl 1 | Ctrl 2 | #1 | #2 |
| Periportal ± Bridging Necrosis | | | | | |
| None | 0 | | | | |
| Mild piecemeal necrosis | 1 | | | 1 | 1 |
| Moderate piecemeal necrosis | 3 | 3 | | | |
| (involves <50% of the circumference | | | | | |
| of most portal tracts) | | | | | |
| Marked piecemeal necrosis | 4 | | 4 | | |
| (involves >50% of the circumference | | | | | |
| of most portal tracts) | | | | | |
| Moderate piecemeal necrosis | 5 | | | | |
| plus bridging necrosis | | | | | |
| Marked piecemeal necrosis | 6 | | | | |
| plus bridging necrosis | | | | | |
| Multilobular necrosis | 10 | | | | |
| Intralobular Degeneration | | | | | |
| and Focal Necrosis | | | | | |
| None | 0 | | | | |
| Mild (acidophilic bodies, ballooning | 1 | | | 1 | 1 |
| degeneration and/or scattered | | | | | |
| foci of hepatocellular necrosis | | | | | |
| in 1/3 of lobules or nodules) | | | | | |
| Moderate (involvement of | 3 | 3 | 3 | | |
| 1/3-2/3 of lobules or nodules) | | | | | |
| Marked (involvement of >2/3 | 4 | | | | |
| of lobules or nodules) | | | | | |
| Portal Inflammation | | | | | |
| No portal inflammation | 0 | | | | |
| Mild (sprinkling of inflammatory | 1 | | | 1 | 1 |
| cells in <1/3 of portal tracts) | | | | | |
| Moderate (increased inflammatory | 3 | 3 | 3 | | |
| cells in 1/3-2/3 of portal tracts) | | | | | |
| Marked (dense packing | 4 | | | | |
| of inflammatory cells | | | | | |
| in >2/3 of portal tracts) | | | | | |
| Fibrosis | | | | | |
| No fibrosis | 0 | | | | |
| Fibrous portal expansion | 1 | | | 1 | 1 |
| Bridging Fibrosis (portal-portal | 3 | 3 | 3 | | |
| or portal-central linkage) | | | | | |
| Cirrhosis | 4 | | | | |
| Total | | 12 | 13 | 4 | 4 |

A novel therapeutic approach was developed that enhanced decomposition of fibrotic tissue and induced regeneration of functional hepatocytes in liver by delivery of M2-specific macrophages into damaged liver. Through portal vein injection of M2 macrophages, which can turn off inflammatory responses by producing various anti-inflammatory cytokines and function in wound healing and tissue repair, significant effects in reduction of liver fibrosis were observed using a well-established carbon tetrachloride administration model. The results of this study demonstrate the utility of administration of M2-specific macrophages to cirrhotic liver to reverse liver fibrosis in afflicted individuals compared to other macrophage types.

### Example 2

### Genetically-Engineered Macrophages

Genetically-engineered M2-specific macrophages are constructed to augment their ability to reverse fibrosis.

To further increase the efficacy of the approach shown in Example 1, M2-specific macrophages are augmented by exogenous expression of collagen targeting agents or collagen receptors, such ITGA-1. Normal M2-specific macrophages are otherwise incapable of attachment or homing to the collagen-rich environment in fibrotic tissue, and expression of ITGA-1 or other collagen targeting agent will likely greatly enhance the retention of the cells to fibrotic tissues and increase the specificity and safety of the approach. Additionally, expression of collagenase (MMP1) in M2-specific macrophages increases the capability of engineered M2 cells to degrade surrounding abnormal collagen matrices and enhance tissue regeneration. MMP1a is not present in the unmodified M2 cells, and it is the major enzyme that degrades collagen in vivo.

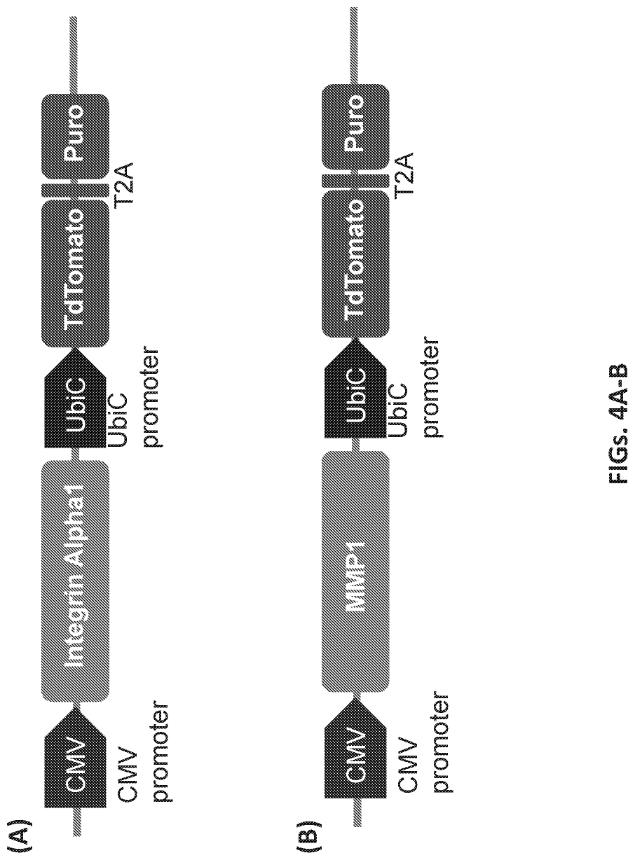

Genetic Constructs. Lentiviral constructs are assembled for the expression of integrin A1 (SEQ ID NO: 1 or SEQ ID NO: 2, FIG. 4A) or MMP1 (SEQ ID NO: 3 or SEQ ID NO: 4, FIG. 4B). Each vector encodes integrin A1 or MMP1 (or MMP1a) driven by a CMV promoter and a selection marker (fluorescence protein tdTomato and puromycin resistant gene, Puro) driven by a constitutive promoter UbiC (Ubiquitin C promoter). TdTomato and Puro are separated by a self-cleavable peptide T2A.

Macrophages. M2-specific macrophages are transfected with one or both lentiviral constructs and selected for incorporation of the expression vector(s) and expression of the recombinant genes.

It is contemplated that recombinant M2-specific recombinant macrophages expressing integrin A1, MMP1 or MMP1a, or both integrin A1 and MMP1 or MMP1a can be introduced into an individual as a novel therapeutic approach for liver fibrosis and other fibrotic diseases. Once introduced, the integrin A1-expressing M2-specific macrophages are localized to the fibrotic lesions with greater specificity and are retained longer than in other tissues due to integrin A1 expression. The MMP1- or MMP1a-expressing recombinant M2-specific macrophages reduce fibrotic lesions at a greater rate than non-recombinant M2-specific macrophages. Integrin A1 and MMP1 expressing M2-specific macrophages demonstrate greater fibrotic lesion removal than either of the singly recombinant M2-specific macrophages and greater than non-recombinant M2-specific macrophages. Such recombinant M2-specific macrophages are useful as cellular therapy products for treating fibrotic diseases.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention.

### Example 3

### Reduction of Cardiac Fibrosis in an Animal Model

In this example, M2-specific macrophages were used to treat an animal model of cardiac fibrosis to demonstrate the ability of the macrophages to ameliorate cardiac fibrosis.

Animal: 12-week old male C57/BL6 mice.

Myocardial Infarction(MI): MI was induced through thoracotomy following permanent ligation of left anterior descending (LAD) coronary artery using a 7-0 suture following the procedure as previously described (19).

Engineered Macrophages Engraftment: 5×10⁵ bone marrow derived M0 macrophage (21) in 0.1 ml PBS were directly injected with a 28-gauge syringe to the border-zone of the infarct site immediately after the ligation. Infarct site was identified by the blanching of left ventricle. Control group was injected with PBS only.

Echocardiography: Echocardiography was performed at 7, 14, and 21 days post operations using a VisualSonic Vevo770 High Resolution Ultrasound System. M-Mode was recorded and echocardiographic parameters were calculated using the pre-installed software in the Vevo770 system.

Tissue Collection: Mice were sacrificed 21 days post surgery. Heart weight and tibia length were measured.

The studies to investigate the engineered macrophages in the treatment of cardiac fibrosis involves 5 steps: (i) Generation of murine myocardial infarction(MI)-induced cardiac fibrosis model using LAD; (ii) Differentiation of bone marrow monocytes into M0 macrophages; (iii) On-site injection of the resulting macrophages into border zone of the infarcted myocardium; (iv) Evaluation of cardiac functions using echocardiography by measuring following parameters: (a) Ejection Fraction (EF)—For left ventricular systolic function, (b) Fraction Shortening (FS) for left ventricular diastolic function, (c) Left Ventricular Internal Dimension at End-diastole (LVID;d) for left ventricular chamber size and myocardium remodeling, (d) Left Ventricular Internal Dimension at End-systole (LVID;s) for left ventricular chamber size and myocardium remodeling; (v) Examination of hypertrophy/myocardium remodeling by measuring heart weight.

The data showed that engineered macrophages successfully improved cardiac performance in mice with myocardial infarction (MI), indicating a cardioprotective effect of engineered macrophages in treating MI-induced cardiac fibrosis and heart failure. The study demonstrated that the engineered macrophages treatment has various advantages over existing therapies: (1) Effectively repressing the development of cardiac fibrosis evidenced by the improved cardiac functions; (2)Developing a novel tissue-specific strategy by using a direct and localized delivery method; and (3) Avoiding side effects induced by existing pharmacological agents.

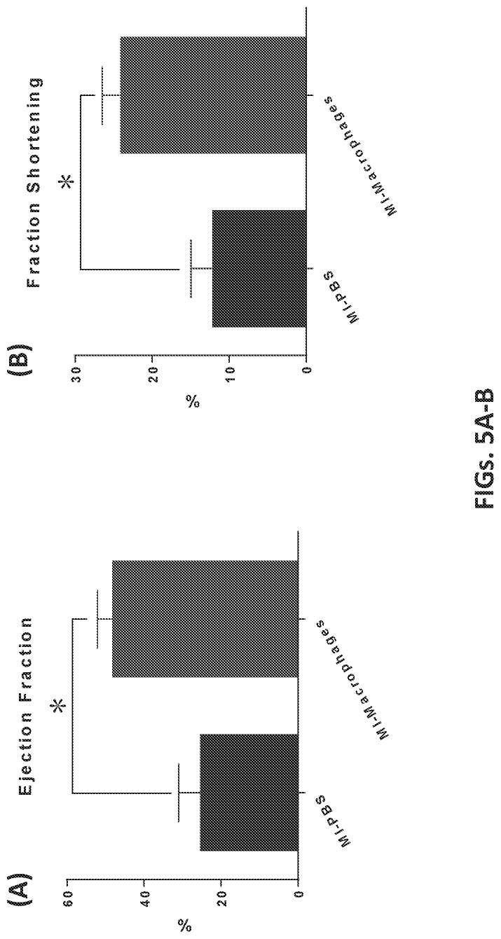

FIG. 5 shows engraftment of engineered macrophages partially prevented MI-induced systolic dysfunction in left ventricle. There were marked deteriorations in Ejection Fraction(EF) and Fraction Shortening(FS) in MI mice received PBS injections, indicating an impaired systolic function/heart failure induced by LAD surgery; Ejection Fraction (EF) and Fraction Shorting (FS) in mice received engineered macrophage were higher than those received PBS, showing cardioprotective effect of engineered macrophage in post-MI heart. Ejection Fraction(EF) and Fraction Shortening(FS) are the two key parameters that measures the percentage of blood pumped out of a filled ventricle with each heartbeat. Decrease in EF and FS indicates the left ventricle loses its ability to distribute enough blood flow to meet the body's needs, a symptom that is clinically defined as “systolic dysfunction”, which ultimately leads to heart failure without effective intervention.

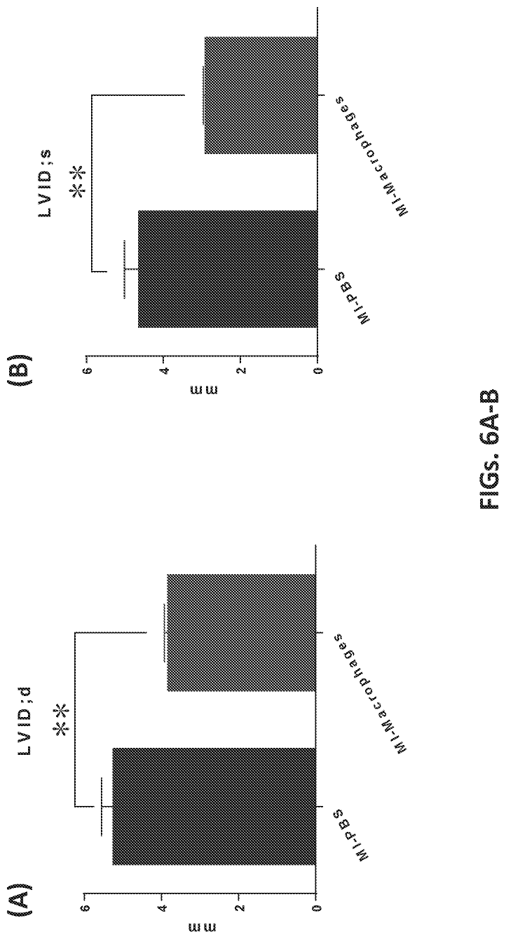

FIG. 6. shows that cellular therapy using engineered macrophages prevented MI-induced LV dilation. Enlargement of LV chamber size was observed following surgical ligation of the LAD in PBS group. Engraftment of engineered macrophages prevented LV from MI-induced dilation. LVID;d and LVID;s are parameters used to measure the internal dimension of the left ventricle at end-diastolic or end-systolic stage of a heart beating cycle. Increase of these two parameter indicates a enlarged left ventricle in a dilated heart caused by pathological myocardium re-construction.

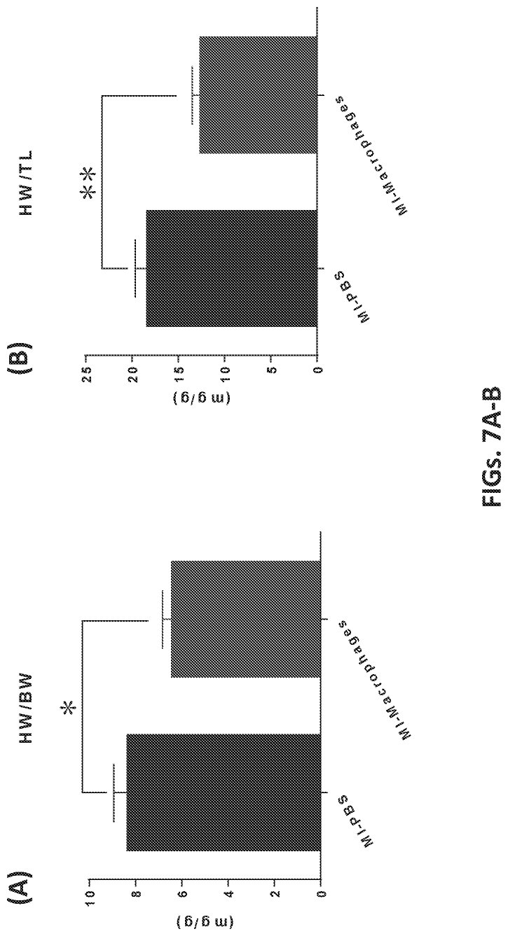

FIG. 7. Shows that cellular therapy using engineered macrophages prevented ischemic myocardium remodeling. Myocardial infarction induced myocardium remodeling in PBS group, evidenced by an increase in heart weight. Lower heart weight in the engineered macrophages group indicates the cellular therapy regressed the remodeling progress. Measurements of “heart weight/body weight” or “heart weight/tibia length” both serve as markers for cardiac fibrosis-induced hypertrophy, as the heart mass increases during the remodeling process.

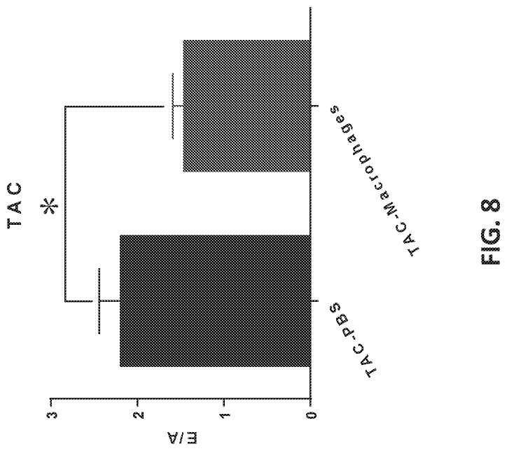

FIG. 8. Shows that cellular therapy using engineered macrophages prevented TAC-induced LV diastolic dysfunction. Increasing of E/A was observed following surgical constraining of the aorta in PBS group. Engraftment of engineered macrophages prevented LV from TAC-induced diastolic dysfunction. E/A is a key parameters used to evaluate the diastolic function of the left ventricle by measuring the peak velocity of mitral annular motion ratio. Increase of this parameter indicates a fibrosis-induced diastolic dysfunction.

In this study, the effectiveness of engineered macrophages has been validated in treating cardiac fibrosis, making this therapeutic approach a competitive candidate that will likely have tremendous potential for clinical applications. The animal results demonstrates a proof-of-principle for the use of engineered macrophages for treating cardiac fibrosis.

### Example 4

### Reduction of Lung Fibrosis in an Animal Model

In this example, M2-specific macrophages were used to treat an animal model of lung fibrosis to demonstrate the ability of the macrophages to ameliorate lung fibrosis.

Bleomycin(BLM)-induced mouse IPF model. The model of BLM-induced lung fibrosis represents the most commonly applied experimental model. BLM is a chemotherapeutic antibiotic that has been identified as a pro-fibrotic agent when lymphoma patients developed pulmonary fibrosis after intravenous administration of BLM. The recognition that bleomycin could result in pulmonary fibrosis in humans led to its use in experimental models, and for four decades it has been the most commonly applied model of experimental lung fibrosis. It is believed that BLM acts by causing single and double-strand DNA breaks in tumor cells and thereby interrupting cell cycle leading to apoptosis.

Animal: 10-week old male C57/BL6 mice.

Generating murine pulmonary fibrosis model: Mice were anesthetized using isoflurane inhalation, then were exposed to bleomycin(BLM) via intratracheal delivery at a dose of 3 U/kg. Control group were administrated with PBS instead.

Isolation and culturing of macrophages: Isolate then differentiate of mouse bone marrow monocytes into M0 macrophages.

Engineered Macrophages Engraftment: 5×10⁶ bone marrow derived M0 macrophage in 0.1 ml PBS were directly injected with an 1 ml insulin syringe via tail vein 7 days after the BLM exposure. Control group was injected with PBS only.

Tissue Collection and Histology Analysis: Mice were sacrificed 14 days post BLM exposure. The lung tissues were fixed for 2 h by the intratracheal instillation of 10% neutral formalin and then removed and continuously fixed for 24 h. Then the tissues were embedded with paraffin and subjected to H&E staining.

The histology analysis showed that Engraftment of macrophages reduced the BLM-induced lung fibrosis and inflammation, and partially preserved structure of pulmonary vesicles.

In this study, a mouse pulmonary fibrosis model is established through intratracheal delivery of bleomycin(BLM). 14 days post the original exposure of BLM, lung tissue affected with inflammatory reactions and suffered a severe destruction of basic structure of pulmonary vesicles.

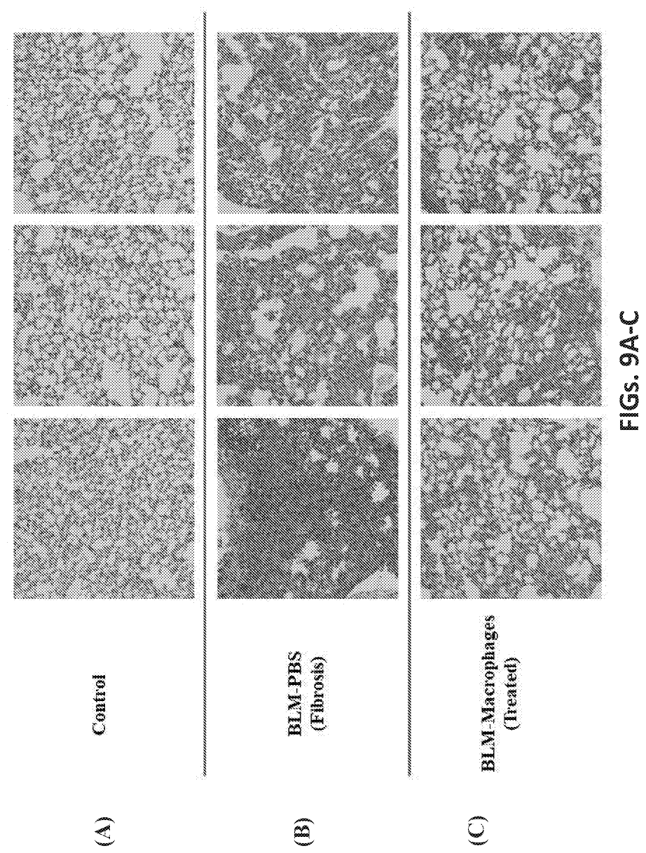

Macrophages treatment via tail vein injection reduced the fibrosis and the degree of inflammation in lungs of mice challenged with BLM. Our data indicate that treatment of macrophages constitute an effective cellular vehicle for the treatment of fibrotic lung disease and present a novel therapeutic approach. The effect of macrophage engraftment on BLM-induced lung injury in mice is shown in FIG. 9. H&E staining on tissue sections prepared from the lungs of C57BL6 mice 14 days after PBS/BLM exposure. FIG. 9A shows the histology of control mice exposed to PBS and injected with PBS. FIG. 9B shows the histology of mice in the fibrosis group exposed to BLM then injected with PBS. FIG. 9C shows the histology of mice in treatment group exposed to BLM then injected with macrophages.

1. A genetically-engineered macrophage, comprising:
a recombinant extracellular matrix (ECM) targeting protein; and/or
a recombinant protease.

2. The genetically-engineered macrophage of claim 1, wherein the recombinant targeting protein is a collagen receptor or a subunit thereof.

3. The genetically-engineered macrophage of claim 2, wherein the collagen receptor or a subunit thereof comprises one or more of an integrin, a discoidin domain receptor, a mannose family receptor, and an immunoglobulin-like receptor.

4. The genetically-engineered macrophage of claim 3, wherein the integrin is an α1β1, α2β1, α10β1, and/or α11β1 integrin.

5. The genetically-engineered macrophage of claim 3, wherein the discoidin domain receptor is DDR1 and/or DDR2.

6. The genetically-engineered macrophage of claim 3, wherein the mannose family receptor is M-phospholipase A2 receptor and/or Endo180.

7. The genetically-engineered macrophage of claim 3, wherein the immunoglobulin-like receptor is glycoprotein VI.

8. The genetically-engineered macrophage of claim 1, wherein the recombinant targeting protein is ITGA-1.

9. The genetically-engineered macrophage of claim 1, wherein the recombinant protease is a matrix metalloproteinase (MMP).

10. The genetically-engineered macrophage of claim 9, wherein the matrix metalloproteinase is MMP1, MMP1a, MMP2, MMP3, MMP7, MMP8, MMP9, MMP10, MMP12, MMP13, MMP14, MMP17, MMP19, MMP20, MMP21, MMP22, MMP24, MMP25, MMP26, MMP27, and/or MMP28.

11. The genetically-engineered macrophage of claim 10, wherein the matrix metalloproteinase is MMP1a.

12. The genetically-engineered macrophage of claim 1, wherein the macrophage is an M2-specific macrophage.

13. The genetically-engineered macrophage of claim 1, wherein the recombinant targeting protein is a human integrin α1 encoded by SEQ ID NO: 3, the recombinant catalytic enzyme is a human MMP1 encoded by SEQ ID NO: 5, and wherein the macrophage is a human M2-specific macrophage.

14. A population of cells comprising the genetically-engineered macrophage of any of the preceding claims.

15. A cellular therapy product, comprising:
a genetically-engineered macrophage comprising at least one of a recombinant extracellular matrix (ECM) targeting protein and a recombinant protease.

16. The cellular therapy product of claim 15 further comprising one or more cell media components and/or therapeutic compounds.

17. The cellular therapy product of claim 16 further comprising an effective amount of one or more of α-tocopherol, interferon-γ, quercetin, an ACE inhibitor, and PPAR-δ.

18. The cellular therapy product of claim 17 further comprising a pharmaceutical reagents and/or excipients suitable for therapeutic application.

19. A method of treating an individual for fibrosis, comprising administering the cellular therapy product according to any of claims 15-18.

20. The method of claim 19, wherein the fibrosis is liver fibrosis, cardiac fibrosis, or lung fibrosis.

21. The method of claim 19, wherein the cellular therapy product is administered by injection to the individual.

22. The method of claim 19, wherein the cellular therapy product is injected in a fibrotic lesion.

23. The method of claim 21, wherein the cellular therapy product comprises of genetically-engineered macrophages was derived from the individual.

24. A method of reversing liver fibrosis in an individual in need thereof, comprising:
administering to the individual a genetically-engineered M2 macrophage capable of expressing recombinant ITGA-1 and MMP1 or MMP1a;
targeting the macrophage to the liver of the individual; and
reversing fibrosis within the liver.

25. A method of treating cardiac fibrosis in an individual in need thereof, comprising:
administering to the individual a genetically-engineered M2 macrophage capable of expressing recombinant ITGA-1 and MMP1 or MMP1a;
targeting the macrophage to the cardiac fibrosis of the individual; and
ameliorating fibrosis within the cardiac tissue.

26. A method of treating lung fibrosis in an individual in need thereof, comprising:
administering to the individual a genetically-engineered M2 macrophage capable of expressing recombinant ITGA-1 and MMP1 or MMP1a;
targeting the macrophage to lung fibrosis of the individual; and
ameliorating fibrosis within the lung tissue.


SEQUENCE LISTING (8 sequences)

SEQ ID NO: 1 - DNA, 3537 bp, Mus musculus
  CDS (1)..(3537)
atggtccccaggcgtcctgctagcctagaggtcactgtagcctgcatctggcttctcaccgttatcctaggcgtctgcatctccttcaacgttgatgtcaaaaactccatgagcttcagtggtccagtggaggacatgtttggatacaccgttcaacagtatgaaaacgaagaaggcaaatgggttcttattggttctcctttagttggccaacccaaagcaagaactggagatgtctataagtgtccagttgggagagagagatcaatgccttgtgtgaagttggatttaccagttaacacatcgattcctaatgtcacagaaataaaggaaaacatgacatttggatcaactttagtcaccaatccgaagggaggatttctggcatgtgggcccttgtatgcctatagatgtggacatttgcattatacgactggcatatgttctgatgtcagccctacatttcaagttgtgaactcgttcgcccctgtacaagaatgtagcacacagcttgacatagtcatcgtcctagatggctccaacagcatctacccctgggaaagtgtcactgcctttttaaacgaccttcttaagaggatggacattggccccaaacagacacaggttggaattgtacaatatggagcaaatgtaacacatgagttcaacctcaataagtattcatccacagaagaggtcctggtggcagcaaacaaaataggccgcaggggtggtctccagacgatgacagcccttggaatagacacagccaggaaagaggcattcactgaagctcggggggccagaaggggagtgaaaaaagtcatggttattgtgactgatggagaatcgcatgacaactatcgcctgaaacaggtcatccaagactgtgaggatgaaaacattcagcgattttccatagctatccttggccactataacagggggaacttaagcaccgaaaaatttgtggaggagataaaatcaatcgcaagtgagcccaccgaaaagcacttcttcaacgtctcagatgagttggccctggtcactattgttaaagctctgggagaaaggatatttgctttggaagctacagctgaccagtcagcagcttcgtttgaaatggagatgtctcagaccggcttcagtgctcattattcacaggactgggtcatgcttggagctgttggagcctatgactggaacggaactgtggtcatgcagaaggctaaccagattgtcatccctcataacaccacctttcaaactgagcccaccaagatgaacgagcctcttgcttcttacttgggttacacagtgaactctgccaccatcccgggagatgtgctctatatcgctgggcagcctcggtacaatcacaccggccaggtcgtcatctacaagatggaggatggggacgtcaacattctgcagacactcagtggagagcagatcggatcctactttggtagcgtcttaaccacaattgacatcgacaaagattcttacactgatctgcttctcgtcggggctcccatgtatatggggacagagaaagaggaacagggcaaggtgtacgtgtacgctgtgaatcagacgaggtttgaatatcaaatgagcctggaaccaattaagcagacctgctgttcatccctgaaggataattcatgcacaaaagaaaacaagaatgagccctgtggggcccgttttggaacagcagttgctgctgtaaaagacctcaacgtggatggctttaatgacgtcgtgattggagctcccctggaagacgaccacgcaggagctgtgtacatttatcatggcagtggcaagaccataaggaaagagtatgcgcaacgcattccctcaggtggggatgggaagacgctgaaattttttggccagtctatccacggagagatggatttaaatggcgatggtctgactgacgtgaccattggaggccttggaggagctgccctcttctgggccagagatgtggctgtagttaaagtgaccatgaattttgaacccaacaaagtgaatattcaaaagaaaaactgccgtgtggagggcaaagaaacagtatgcataaatgctacaatgtgttttcatgtgaaattgaagtctaaagaagactcagtttacgaggctgatctgcagtaccgtgtcacccttgattcgctgaggcagatatcacggagctttttttctggaactcaggaaaggaggattcaaagaaacctcaccgttcgagaatccgaatgcatcaggcactccttctacatgttggataagcacgactttcaggactccgtgagagtgactttggattttaacctcactgatccagaaaatgggcccgtgctcgatgacgctctgccaaactcagtccatggacatattccttttgccaaagactgtgggaacaaggaaagatgcgtttcagacctcaccctggatgtgtccacaacagaaaagaacctgctgattgtcagatcccagaatgacaagttcaatgtcagcctcaccgtcaaaaacaagggagacagtgcgtacaacacccggacagtggttcagtattctccaaatctgattttttcaggaattgaggagatccaaaaagatagctgcgaatccaatcaaaatatcacgtgtagagttggatatcctttcctgaggacaggagacgtggttaacttcaaaataatattccagtttaatacatcacatctctcagaaaatgcaattattcatttaagtgcaacaagtgacagtgaagagcccctggaatctctttacgataatgaagtaaatatttccatccccgtaaaatatgaagtcggactgcagttttacagttctgcgagtgaacaccacatttcagttgctgccaatgagactgttcctgagctgattaattccaccaaggacattggggatgaaattaatgtcttctacacgattagaaaaagagggcatttcccaatgccagaacttcggctggcaatttcatttcccaatctgacgtcagatggctatcctgtactgtacccaactggatggtcatcttctgataatgtaaactgcagacctcgaagccttgaggaccctttgggtatcaactctgggaagaaaatgacaatatcaaagtctgaggttctcaaaagaggcacaatccaggactgcagtacctgcaagattgctaccatcacgtgtcatctccttccctcggatgtgagtcaagtgaatgtctcactcatcttgtggaaaccaactttcataaaagcacatttttccagcttaaatcttaccatacggggagaacttcagagtgaaaactcatcgctgactttaagcagcagcaaccggaaacgagagctggctattcagatatccaaagacgggctcccaggccgagtgccgctgtgggttatcctcctgagcgcctttgcgggactcctgctgttaatgctgcttatattagctctgtggaagattggattcttcaaaaggccactaaagaagaaaatggagaaa

SEQ ID NO: 2 - protein, 1179 aa, Mus musculus
MVPRRPASLEVTVACIWLLTVILGVCISFNVDVKNSMSFSGPVEDMFGYTVQQYENEEGKWVLIGSPLVGQPKARTGDVYKCPVGRERSMPCVKLDLPVNTSIPNVTEIKENMTFGSTLVTNPKGGFLACGPLYAYRCGHLHYTTGICSDVSPTFQVVNSFAPVQECSTQLDIVIVLDGSNSIYPWESVTAFLNDLLKRMDIGPKQTQVGIVQYGANVTHEFNLNKYSSTEEVLVAANKIGRRGGLQTMTALGIDTARKEAFTEARGARRGVKKVMVIVTDGESHDNYRLKQVIQDCEDENIQRFSIAILGHYNRGNLSTEKFVEEIKSIASEPTEKHFFNVSDELALVTIVKALGERIFALEATADQSAASFEMEMSQTGFSAHYSQDWVMLGAVGAYDWNGTVVMQKANQIVIPHNTTFQTEPTKMNEPLASYLGYTVNSATIPGDVLYIAGQPRYNHTGQVVIYKMEDGDVNILQTLSGEQIGSYFGSVLTTIDIDKDSYTDLLLVGAPMYMGTEKEEQGKVYVYAVNQTRFEYQMSLEPIKQTCCSSLKDNSCTKENKNEPCGARFGTAVAAVKDLNVDGFNDVVIGAPLEDDHAGAVYIYHGSGKTIRKEYAQRIPSGGDGKTLKFFGQSIHGEMDLNGDGLTDVTIGGLGGAALFWARDVAVVKVTMNFEPNKVNIQKKNCRVEGKETVCINATMCFHVKLKSKEDSVYEADLQYRVTLDSLRQISRSFFSGTQERRIQRNLTVRESECIRHSFYMLDKHDFQDSVRVTLDFNLTDPENGPVLDDALPNSVHGHIPFAKDCGNKERCVSDLTLDVSTTEKNLLIVRSQNDKFNVSLTVKNKGDSAYNTRTVVQYSPNLIFSGIEEIQKDSCESNQNITCRVGYPFLRTGDVVNFKIIFQFNTSHLSENAIIHLSATSDSEEPLESLYDNEVNISIPVKYEVGLQFYSSASEHHISVAANETVPELINSTKDIGDEINVFYTIRKRGHFPMPELRLAISFPNLTSDGYPVLYPTGWSSSDNVNCRPRSLEDPLGINSGKKMTISKSEVLKRGTIQDCSTCKIATITCHLLPSDVSQVNVSLILWKPTFIKAHFSSLNLTIRGELQSENSSLTLSSSNRKRELAIQISKDGLPGRVPLWVILLSAFAGLLLLMLLILALWKIGFFKRPLKKKMEK

SEQ ID NO: 3 - DNA, 3537 bp, Homo sapiens
  CDS (1)..(3537)
atggcccctcggccccgcgcccgcccaggggtcgctgtcgcctgctgctggctcctcactgttgttctacgctgctgcgtatcattcaatgttgatgtgaaaaattcaatgactttcagcggcccggtggaagacatgtttggatatactgttcaacaatatgaaaatgaagaaggaaaatgggtgcttattggttctccgttagttggccaacccaaaaacagaactggagatgtctataagtgtccagttgggagaggtgaatcattaccttgtgtaaagttggatctaccagttaatacatcaattcccaatgtcacagaagtaaaggagaacatgacatttggatcaactttagtcaccaacccaaatggaggatttctggcttgtgggcccttatatgcctatagatgtggacatttgcattacacaactggaatctgttctgacgtcagccccacatttcaagtcgtgaattccattgcccctgtacaagaatgcagcactcaactggacatagtcatagtgctggatggttccaacagtatttacccatgggacagtgttacagcttttttaaatgaccttcttgaaagaatggatattggtcctaaacagacacaggttggaattgtacagtatggagaaaacgtgacccatgagttcaacctcaataagtattcttccaccgaagaggtacttgttgcagcaaagaaaatagtccagagaggtggccgccagactatgacagctcttggaatagacacagcaagaaaggaggcattcacggaagcccggggtgcccgaagaggagttaaaaaagtcatggttattgtgacagatggagagtctcatgacaatcatcgactgaagaaggtcatccaagactgtgaagatgaaaacattcaacggttttccatagctattcttggcagctataaccgaggaaatttaagcactgaaaaatttgtggaggaaataaaatcaattgcaagtgaacccactgaaaagcatttcttcaatgtctctgatgaattggctctagtcaccattgttaaaactctgggagaaagaatatttgccctggaagccacagctgaccagtcagcagcttcatttgaaatggaaatgtctcagactggcttcagtgctcattattcacaggactgggtcatgcttggagcagtaggagcctatgattggaatggaacagttgtcatgcagaaggctagtcaaatcataatccctcgaaacacaacctttaatgttgagtctaccaaaaagaatgaaccgcttgcttcttatttaggttacactgtaaactctgctactgcttcttctggagatgtgctctatattgctggacagcctcggtacaatcatacaggccaggtcattatctacaggatggaagatggaaacatcaaaattctccagacgctcagtggagaacagattggttcctactttggcagtattttaacaacaactgacattgacaaggattctaatactgacattcttctagtcggagcccctatgtacatgggaacagagaaggaggagcaaggaaaagtgtatgtgtatgctctcaatcagacaaggtttgaatatcaaatgagcctggaacctattaagcagacgtgctgttcatctcggcagcacaattcatgcacaacagaaaacaaaaatgagccatgcggggctcgttttggaactgcaattgctgctgtaaaagacctcaatcttgatggatttaatgacatcgtgataggagctccgctggaagatgatcacgggggagctgtgtacatttatcatggaagtggcaagactataaggaaagagtatgcacaacgtattccatcaggtggggatggtaagacactgaaattttttggccagtctatccacggagaaatggatttaaatggtgacggtctgacagatgtgactattgggggccttggtggtgctgccctcttctggtcccgagatgtggccgtagttaaagtgaccatgaattttgagccaaataaagtgaatattcaaaagaaaaactgccatatggagggaaaggaaacagtatgcataaatgctacagtgtgttttgatgtgaaattaaagtctaaagaagacacgatttatgaagctgatttgcagtaccgtgtcaccctagattcactaagacaaatatcacgaagttttttctctggaactcaagagagaaaggttcaaaggaacatcacagttcgaaaatcagaatgcactaagcactccttctacatgttggacaagcatgactttcaggactctgtgagaataacgttggactttaatcttaccgatccagaaaatgggcctgttcttgatgattctctaccaaactcagtacatgaatatattccctttgccaaagattgtggaaataaggaaaaatgtatctcagacctcagcctgcatgtcgccaccactgaaaaggacctgctgattgtccgatcccagaatgataagttcaacgttagcctcacagtcaaaaatacaaaggacagtgcctataacaccaggacaatagtgcattattctccaaatctagttttttcaggaattgaggctatccaaaaagacagttgtgaatctaatcataatatcacatgtaaagttggatatcccttcctgagaagaggagagatggtaactttcaaaatattgtttcagtttaacacatcctatctcatggaaaatgtgaccatttatttaagtgcaacaagtgacagcgaagaacctcctgaaaccctttctgataatgtagtaaacatttctatcccggtaaaatatgaagttggactacagttttacagctctgcaagtgaataccacatttcaattgctgccaatgagacagtccctgaagttattaattctactgaggacattggaaatgaaattaatatcttctacttgattagaaaaagtggatcttttccaatgccagagcttaagctgtcaatttcattccccaatatgacatcaaatggttaccctgtgctgtacccaactggattgtcatcttctgagaatgcaaactgcagaccccatatctttgaggatcctttcagtatcaactctggaaagaaaatgactacatcaactgaccatctcaaacgaggcacaattctggactgcaatacatgtaaatttgctaccatcacatgtaatctcacttcttctgacatcagccaagtcaatgtttcgcttatcttgtggaaaccaacttttataaaatcatatttttccagcttaaatcttactataaggggagaacttcggagtgaaaatgcatctctggttttaagtagcagcaatcaaaaaagagagcttgctattcaaatatccaaagatgggctaccgggcagagtgccattatgggtcatcctgctgagtgcttttgccggattgttgctgttaatgctgctcattttagcactgtggaagattggattcttcaaaagaccactgaaaaagaaaatggagaaa

SEQ ID NO: 4 - protein, 1179 aa, Homo sapiens
MAPRPRARPGVAVACCWLLTVVLRCCVSFNVDVKNSMTFSGPVEDMFGYTVQQYENEEGKWVLIGSPLVGQPKNRTGDVYKCPVGRGESLPCVKLDLPVNTSIPNVTEVKENMTFGSTLVTNPNGGFLACGPLYAYRCGHLHYTTGICSDVSPTFQVVNSIAPVQECSTQLDIVIVLDGSNSIYPWDSVTAFLNDLLERMDIGPKQTQVGIVQYGENVTHEFNLNKYSSTEEVLVAAKKIVQRGGRQTMTALGIDTARKEAFTEARGARRGVKKVMVIVTDGESHDNHRLKKVIQDCEDENIQRFSIAILGSYNRGNLSTEKFVEEIKSIASEPTEKHFFNVSDELALVTIVKTLGERIFALEATADQSAASFEMEMSQTGFSAHYSQDWVMLGAVGAYDWNGTVVMQKASQIIIPRNTTFNVESTKKNEPLASYLGYTVNSATASSGDVLYIAGQPRYNHTGQVIIYRMEDGNIKILQTLSGEQIGSYFGSILTTTDIDKDSNTDILLVGAPMYMGTEKEEQGKVYVYALNQTRFEYQMSLEPIKQTCCSSRQHNSCTTENKNEPCGARFGTAIAAVKDLNLDGFNDIVIGAPLEDDHGGAVYIYHGSGKTIRKEYAQRIPSGGDGKTLKFFGQSIHGEMDLNGDGLTDVTIGGLGGAALFWSRDVAVVKVTMNFEPNKVNIQKKNCHMEGKETVCINATVCFDVKLKSKEDTIYEADLQYRVTLDSLRQISRSFFSGTQERKVQRNITVRKSECTKHSFYMLDKHDFQDSVRITLDFNLTDPENGPVLDDSLPNSVHEYIPFAKDCGNKEKCISDLSLHVATTEKDLLIVRSQNDKFNVSLTVKNTKDSAYNTRTIVHYSPNLVFSGIEAIQKDSCESNHNITCKVGYPFLRRGEMVTFKILFQFNTSYLMENVTIYLSATSDSEEPPETLSDNVVNISIPVKYEVGLQFYSSASEYHISIAANETVPEVINSTEDIGNEINIFYLIRKSGSFPMPELKLSISFPNMTSNGYPVLYPTGLSSSENANCRPHIFEDPFSINSGKKMTTSTDHLKRGTILDCNTCKFATITCNLTSSDISQVNVSLILWKPTFIKSYFSSLNLTIRGELRSENASLVLSSSNQKRELAIQISKDGLPGRVPLWVILLSAFAGLLLLMLLILALWKIGFFKRPLKKKMEK

SEQ ID NO: 5 - DNA, 1407 bp, Homo sapiens
  CDS (1)..(1407)
atgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaat

SEQ ID NO: 6 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 7 - DNA, 1392 bp, Mus musculus
  CDS (1)..(1392)
atgcctagccttcctttgctgttgcttctctgggctgctagctcatacagtttccctgtgtttcacaacggagaccggcaaaatgtggagacagtctggaaatacctggaaaactactacaacttgggcaaaaacatgcaagctaaaaacgtgaatggcaaggagatgatggctgaaaagctgaggcaaatgcagcagttatttgggctgaaagtgactggaaattcagatcctgaaaccctgagagctatgaagaagcccaggtgtggggtgcctgatgtggccccatatgccattactcacaacaatcctcgttggaccaaaacacatctgacatacagcattttaaactacacaccatatttgccaaaagcagttgtggaagatgccatcgcgagagcctttagagtctggagtgatgtgacaccacttacgttccaaagagtctttgaggaggaaggcgatattgtgctctccttccacagaggagaccatggtgacaacaacccatttgatggacctaactataagcttgctcacactttccagccaggcccaggtttggggggtgatgttcattatgaccttgatgagacgtggaccaacagcagtgaaaatttcaacttgttctatgttacggctcatgaactgggtcactcccttgggctcactcattctagtgatataggagcactaatgttccccagttacacgtggtacactgaagactttgtgctaaaccaggatgatattaatcgcatccaggacttatatggaccttccccaaatcccatccagccaacaggtgcaacaacaccacatccatgtaatggtgatctaacctttgatgctataactacatttaggggagaggtgtttttcttcaaaggcaggttctacattcgggtaaatagattcatgccagaacctgagctcaatttaataggtattctctggccaaatcttccagttaaacttgacgctgcttatgaagctagtatgatagatcaagtccgctatttcaaaggcagcaaagtatgggctgttcaagagcagagtgtactgagaggattccccagagacatccacagtttctttggcttccctagcaatgtgacacacattgatgctgctgtttgtgaggaagagactggaaaaacatacttctttgttgaccacatgtactggaggtatgatgaaaatacacagtctatggatccaggttatcccagattaacagcagaagacttccctggaattgatgataaagttgatgatgttttccaaaaaggagaaaatttctatttctttcaccaatcagttcaacacagatttaacctccaaataagaagagttgatgattcccgtgattctagtacatggttcaactgc

SEQ ID NO: 8 - protein, 464 aa, Mus musculus
MPSLPLLLLLWAASSYSFPVFHNGDRQNVETVWKYLENYYNLGKNMQAKNVNGKEMMAEKLRQMQQLFGLKVTGNSDPETLRAMKKPRCGVPDVAPYAITHNNPRWTKTHLTYSILNYTPYLPKAVVEDAIARAFRVWSDVTPLTFQRVFEEEGDIVLSFHRGDHGDNNPFDGPNYKLAHTFQPGPGLGGDVHYDLDETWTNSSENFNLFYVTAHELGHSLGLTHSSDIGALMFPSYTWYTEDFVLNQDDINRIQDLYGPSPNPIQPTGATTPHPCNGDLTFDAITTFRGEVFFFKGRFYIRVNRFMPEPELNLIGILWPNLPVKLDAAYEASMIDQVRYFKGSKVWAVQEQSVLRGFPRDIHSFFGFPSNVTHIDAAVCEEETGKTYFFVDHMYWRYDENTQSMDPGYPRLTAEDFPGIDDKVDDVFQKGENFYFFHQSVQHRFNLQIRRVDDSRDSSTWFNC